(19) United States
(12) Patent Application Publication
(10) Pub. No.: US 2011/0053197 A1
(43) Pub. Date: Mar. 3, 2011

(54) UBIQUITIN PROTEASOME SYSTEM PROFILING AND THE USE THEREOF IN CLINICAL APPLICATIONS FOR CANCER DIAGNOSIS

(75) Inventors: Maher Albitar, Coto De Caza, CA (US); Wanlong Ma, Aliso Viejo, CA (US); Ke Zhang, Thousand Oaks, CA (US); Xi Zhang, Aliso Viejo, CA (US); Xiuqiang Wang, Irvine, CA (US); Kevin Qu, Irvine, CA (US); Anthony Sferruzza, San Clemente, CA (US)

(73) Assignee: Quest Diagnostics Investments Incorporated

(21) Appl. No.: 12/547,460

(22) Filed: Aug. 25, 2009

(51) Int. Cl. C12Q 1/37

(52) U.S. Cl. 435/23

(57) ABSTRACT

Provided herein are methods for the diagnosis, prognosis, or management of neoplastic diseases, i.e. cancer, and other diseases using profiles of the ubiquitin-proteasome system determined from acellular body fluids or cell-containing samples. Further provided are methods of predicting response to therapy in certain populations of cancer patients.

### FIELD OF THE INVENTION

The invention relates to the diagnosis, prognosis, and management of disease, including cancer.

### BACKGROUND OF THE INVENTION

The following discussion of the background of the invention is merely provided to aid the reader in understanding the invention and is not admitted to describe or constitute prior art to the present invention.

The ubiquitin-proteasome system (UPS) is responsible for the degradation of approximately 80-90% of normal and abnormal intracellular proteins and therefore plays a central role in a large number of physiological processes. For example, the regulated proteolysis of cell cycle proteins, including cyclins, cyclin-dependent kinase inhibitors, and tumor suppressor proteins, is required for controlled cell cycle progression and proteolysis of these proteins occurs via the ubiquitin-proteasome pathway (Deshaies, Trends in Cell Biol., 5:428-434 (1995) and Hoyt, Cell, 91:149-151 (1997)). In another example, the activation of the transcription factor NF-KB, which itself plays a central role in the regulation of genes involved in the immune and inflammatory responses, is dependent upon the proteasome-mediated degradation of an inhibitory protein, IκαB-α (Palombella et al., WO 95/25533). In yet another example, the ubiquitin-proteasome pathway plays an essential role in antigen presentation through the continual turnover of cellular proteins (Goldberg and Rock, WO 94/17816).

While serving a central role in normal cellular homeostasis, the UPS also mediates the inappropriate or accelerated protein degradation occurring as a result or cause of pathological conditions including cancer, inflammatory diseases, and autoimmune diseases, characterized by deregulation of normal cellular processes. In addition, the cachexia or muscle wasting associated with conditions such as cancer, chronic infectious diseases, fever, muscle atrophy, nerve injury, renal failure, and hepatic failure results from an increase in proteolytic degradation by the UPS (Goldberg, U.S. Pat. No. 5,340,736 (1994)). Furthermore, the cytoskeletal reorganization that occurs during maturation of protozoan parasites is proteasome-dependent (Gonzales et al., J. Exp. Med., 184:1909 (1996)).

Central to this system is the 26S proteasome, a multi-subunit proteolytic complex, consisting of one 20S proteasome core and two flanking 19S complexes. The 20S proteasome consists of four rings: two outer α-rings and two inner β-rings surrounding a barrel-shaped cavity. The two inner β-rings form a central chamber that harbors the catalytic site for the chymotryptic, tryptic, and caspase-like activities (von Mikecz, J Cell Sci, 119(10):1977-84, 2006).

Proteins targeted for degradation by the proteasome contain a recognition signal. This signal consists of a polyubiquitin chain that is selectively attached to the protein target by the sequential addition of ubiquitin monomers. The polyubiquitin signal is recognized by the 19S complex, which mediates the entry of the target protein into the proteolytic chamber.

### SUMMARY OF THE INVENTION

The present invention is based on the discovery that the specific activity of proteasomal peptidases may be detected in patient samples and that such activity can have clinical value in the diagnosis and prognosis of certain disease states.

In one aspect, the invention provides methods for diagnosing a neoplastic disease in a subject, the method comprising: determining, in a body fluid sample (e.g., an acellular body fluid sample) obtained from the subject, the specific activity of one or more (i.e., one, two, or three) proteasomal peptidases selected from the group consisting of chymotrypsin-like activity (Ch-L), trypsin-like activity (Tr-L), and caspase-like activity (Cas-L), wherein the specific activity is determined by normalizing the one or more peptidase activities to the amount of proteasomal protein in the sample, and wherein a difference of the specific activity of one or more proteasomal peptidases compared to a reference specific activity indicates a neoplastic disease in the subject. In one embodiment, the acellular body fluid is selected from the group consisting of serum and plasma.

In certain embodiments, an increase or decrease in the specific activity of one or more proteasomal peptidases relative to the corresponding specific activity in a comparable sample from one or more healthy individuals is a factor favoring diagnosis of a neoplastic disease, e.g., a carcinoma, a sarcoma, a neuroblastoma, a leukemia, a lymphoma, and a solid tumor. In illustrative embodiments, the methods are used to diagnose hepatocellular carcinoma. In further embodiments, the methods are used to diagnose a leukemia selected from the group consisting of chronic lymphocytic leukemia (CLL), acute myeloid leukemia (AML), and acute lymphoblastic leukemia (ALL).

In suitable embodiments, the determined specific activity is compared to a reference specific activity. In some embodiments, the reference specific activity is the specific activity for each peptidase in a comparable sample from one or more healthy individuals. In a particular embodiment, the reference specific activity is a cutoff value that has been statistically calculated based on the specific activity determined from a particular population of individuals (e.g., a population of cancer patients) or based on a statistical model to determine a cutoff value for predicting a specific clinical behavior. In this embodiment, a determined specific activity greater than or lower than a cutoff value is related to an unfavorable diagnosis for the patient. In some embodiments, a determined specific activity in the patient sample that is the same as or substantially the same as the specific activity in the reference sample (i.e., a comparable acellular body fluid sample from one or more healthy individuals) reflects a positive prognosis for the patient.

In one aspect, the present invention provides a method of diagnosing a neoplastic disease in a subject, the method comprising: determining the amount of proteasomal protein in a test sample for the subject; determining the amount of one or more (i.e., one, two, or three) proteasomal peptidase activities in a test sample from the subject, the peptidase activities selected from the group consisting of chymotrypsin-like activity (Ch-L), trypsin-like activity (Tr-L), and caspase-like activity (Cas-L), normalizing the level of one or more proteasomal peptidase activities to the level of proteasomes to provide a specific activity of the one or more proteasomal peptidases; and using the specific activity of the one or more proteasomal peptidases to diagnose the presence of a neoplastic disease in the subject.

In another aspect, the invention provides a method of determining a prognosis of a subject having a neoplastic disease, wherein the method comprises: determining the specific activity of one or more (i.e., one, two, or three) proteasomal peptidases selected from the group consisting of chymotrypsin-like activity (Ch-L), trypsin-like activity (Tr-L), and caspase-like activity (Cas-L), wherein the specific activity is determined by normalizing the one or more proteasomal peptidases activities to a level of proteasomal protein in the sample, and wherein a difference of the specific activities compared to a reference specific activity indicates the prognosis of the subject having a neoplastic disease. In one embodiment, the prognosis is selected from the group consisting of survival rate, 5-year survival rate, and complete remission duration (CRD).

In one embodiment, the reference specific activity is the specific activity of corresponding proteasomal proteins in a comparable sample from one or more healthy individuals. In one embodiment, the test sample is a cell-containing sample. In another embodiment, the test sample is an acellular body fluid sample, e.g., serum or plasma.

In one aspect, the invention provides a method for diagnosing hepatocellular carcinoma (HCC) in a subject, the method comprising; (a) assaying the amount of one or more (i.e., one, two, or all three) of Ch-L-, Tr-L-, and Cas-L-activity in a sample from the subject; (b) assaying the amount of one or more (i.e., one, two, three, or all four) of alpha-fetoprotein (AFP), AFP-L3, des-gamma-carboxyprothrombin (DCP), and ubiquitin in the sample; (c) assaying the amount of proteasomal protein in a sample from the subject and normalizing one or more of Ch-L, Tr-L, and Cas-L to determine the specific activity (Ch-L/p, Tr-L/p, and Cas-L/p, respectively); (d) determining a score (e.g., a UPS score) for the subject based on the assayed levels in steps (a) and (b); (e) comparing the score to a cut-off value that is predictive of a disease or symptom in order to determine the presence of HCC in the subject.

In one embodiment, the enzymatic activity of each of Cas-L, Tr-L/p, and Ch-L are assayed. In another embodiment, the amount of one or both of AFP and DCP are assayed.

In an illustrative embedment, the score is determined using the algorithm:

Score=y/(1+y)

wherein,

y=exp[−X+(C₁×Age)+(C₂×Gender)+(C₃×DCP)−(C₄×Ch-L)+(C₅×Cas-L/p)+(C₆×Tr-L/p)+(C₇×AFP)]

wherein X is from 16.7293 to 20.4471 inclusive; C₁ is from 0.2027 to 0.2479 inclusive; C₂ is from 3.9908 to 4.8778 inclusive; C₃ is from 0.97557 to 1.1681 inclusive; C₄ is from 23.5331 to 28.7627 inclusive; C₅ is from 3.0299 to 3.7033 inclusive; C₆ is from 0.0558 to 0.0682 inclusive; C₇ is from 0.1534 to 0.1876 inclusive; and wherein, age is provided in years; male gender=1, female gender=0; AFP is reported in ng/mL; DCP is reported in ngmL; Cas-L/p is reported in pmol product/sec/pg proteasome; Tr-L/p is reported in pmol product/sec/pg proteasome; and Ch-L is reported in pmol product/sec/mL. In one embodiment, X is about 18.5882: C₁ is about 0.2253; C₂ is about 4.4343; C₃ is about 1.0619; C₄ is about 26.1479; C₅ is about 3.3666; C₆ is about 0.062; C₇ is about 0.1705.

The determined score may be compared to a cutoff value that has been statistically calculated based on the measurements obtained from a particular population of individuals. In one embodiment, the cut-off value is about 0.5 and a score less than about 0.5 is indicative of the absence of HCC in the subject. In one embodiment, the cut-off value is about 0.5 and a score greater than or equal to about 0.5 is indicative of HCC in the subject. In one embodiment, the score is used for the choice of a suitable treatment for the subject.

In another aspect, the invention provides a method for monitoring progression of hepatocellular carcinoma (HCC) in a subject, the method comprising: (a) providing a first sample from the subject; (b) assaying in the sample the amount of (i) enzymatic activity from one or more of Ch-L, Tr-L, and Cas-L, (ii) one or more of AFP, AFP-L3, DCP, and ubiquitin, (iii) proteasomal protein and normalizing at least one enzymatic activity determined in step (b)(i); (c) determining a score for the subject based on the assayed levels in (b); (d) comparing the score to a cut-off score that is predictive of HCC in order to determine the extent of HCC in the subject; (e) providing a second sample from the subject, wherein the second sample is obtained after the first sample; (f) repeating steps (b) to (d) to determine the extent of HCC as indicated by the second sample; and (g) comparing the extent of HCC indicated by the first sample to the extent of HCC indicated by the second sample, wherein a higher extent of HCC in the second sample in comparison to the first sample indicates progression of HCC or a lesser extent of HCC in the second sample in comparison to the first sample indicates regression of HCC.

### BRIEF DESCRIPTION OF THE FIGURE

FIG. 1 is a series of charts showing AUROC curves comparing the UPS signature model (AFP, DCP, Ch-L, Tr-L/p, and Cas-L/p, age, and gender) with the HCC model (AFP, AFP-L3, DCP, age, and gender) in patients with HCC and those with chronic liver diseases (CLD). FIG. 1A shows the results from 112 patients with HCC vs. 60 with CLD; FIG. 1B shows the results from 44 patients with small tumors (≦3 cm) vs. 60 patients with CLD; and

FIG. 1C shows the results from 68 patients with large tumors (>3 cm) vs. 60 patients with CLD.

### DETAILED DESCRIPTION OF THE INVENTION

The present invention relates generally to methods of assessing the ubiquitin-proteasome system (UPS) for the diagnosis of disease. As demonstrated herein, increasing or decreasing amounts of the specific activity of one or more proteasomal peptidases correlates with the presence of disease or the prognosis of a patient suffering from a disease. In particular, methods for diagnosing neoplastic diseases, determining the likelihood of survival, and methods for predicting likelihood for responsiveness to therapy are provided.

The present technology is described herein using several definitions, as set forth throughout the specification. As used herein, unless otherwise stated, the singular forms “a,” “an,” and “the” include plural reference. Thus, for example, a reference to “a proteasome” is a reference to one or more proteasomes.

The term “about” as used herein in reference to quantitative measurements or values, refers to the enumerated value plus or minus 10%, unless otherwise indicated.

The term “antibody” as used herein encompasses both monoclonal and polyclonal antibodies that fall within any antibody classes, e.g., IgG, IgM, IgA, IgE, or derivatives thereof. The term “antibody” also includes antibody fragments including, but not limited to, Fab, F(ab′)₂, and conjugates of such fragments, and single-chain antibodies comprising an antigen recognition epitope. In addition, the term “antibody” also means humanized antibodies, including partially or fully humanized antibodies. An antibody may be obtained from an animal, or from a hybridoma cell line producing a monoclonal antibody, or obtained from cells or libraries recombinantly expressing a gene encoding a particular antibody.

The terms “assessing” and “evaluating” are used interchangeably to refer to any form of measurement, and include determining if a characteristic, trait, or feature is present or not. The terms “determining,” “measuring,” “assessing,” and “assaying” are used interchangeably and include both quantitative and qualitative determinations. Assessing may be relative or absolute. “Assessing the presence of” includes determining the amount of something present, as well as determining whether it is present or absent.

The term “body fluid” or “bodily fluid” as used herein refers to any fluid from the body of an animal. Examples of body fluids include, but are not limited to, plasma, serum, blood, lymphatic fluid, cerebrospinal fluid, synovial fluid, urine, saliva, mucous, phlegm and sputum. A body fluid sample may be collected by any suitable method. The body fluid sample may be used immediately or may be stored for later use. Any suitable storage method known in the art may be used to store the body fluid sample; for example, the sample may be frozen at about −20° C. to about −70° C. Suitable body fluids are acellular fluids. “Acellular” fluids include body fluid samples in which cells are absent or are present in such low amounts that the peptidase activity level determined reflects its level in the liquid portion of the sample, rather than in the cellular portion. Typically, an acellular body fluid contains no intact cells. Examples of acellular fluids include plasma or serum, or body fluids from which cells have been removed.

The term “clinical factors” as used herein, refers to any data that a medical practitioner may consider in determining a diagnosis or prognosis of disease. Such factors include, but are not limited to, the patient's medical history, a physical examination of the patient, complete blood count, analysis of the activity of enzymes (e.g., liver enzymes), examination of blood cells or bone marrow cells, cytogenetics, and immunophenotyping of blood cells. Specific activity of one or more proteasomal peptidases is a clinical factor.

The term “comparable” or “corresponding” in the context of comparing two or more samples, means that the same type of sample (e.g., plasma) is used in the comparison. For example, a specific activity level of one or more proteasomal peptidases in a sample of plasma can be compared to a specific activity level in another plasma sample. In some embodiments, comparable samples may be obtained from the same individual at different times. In other embodiments, comparable samples may be obtained from different individuals (e.g., a patient and a healthy individual). In general, comparable samples are normalized by a common factor. For example, acellular body fluid samples are typically normalized by volume body fluid and cell-containing samples are normalized by protein content or cell count.

The phrase “cut-off value” as used herein refers to a UPS score that is statistically predictive of a symptom or disease or lack thereof. In a particular embodiment, the cut-off value is about 0.5 and the UPS score distinguishes between HCC and an absence of HCC. For example, a UPS score greater than or equal to a cut-off value of about 0.5 is predictive of HCC. A UPS score less than a cut-off value of about 0.5 is predictive of an absence of HCC. In certain embodiments, this cut-off value may be between 0.425 to 0.575 inclusive, or between 0.450 to 0.550 inclusive, or between 0.475 to 0.525 inclusive. Alternatively, the cut-off value may be 0.425, 0.450, 0.5 0.475, 0.525, 0.550, and even 0.575. The above numbers are subject to 5% variation.

As used herein, the term “diagnosis” means detecting a disease or disorder or determining the stage or degree of a disease or disorder. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition, Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease. The term “diagnosis” also encompasses determining the therapeutic effect of a drug therapy, or predicting the pattern of response to a drug therapy. The diagnostic methods may be used independently, or in combination with other diagnosing and/or staging methods known in the medical art for a particular disease or disorder, e.g., a neoplastic disease.

As used herein, the phrase “difference of the level” refers to differences in the quantity of a particular marker, such as a protein or protein activity, in a sample as compared to a control or reference level. For example, the quantity of particular protein and/or the amount of a protein activity may be present at an elevated amount or at a decreased amount in samples of patients with a neoplastic disease compared to a reference level. In one embodiment, a “difference of a level” may be a difference between the specific activity of a proteasomal peptidase present in a sample as compared to a control of at least about 1%, at least about 2%, at least about 3%, at least about 5%, at least about 10%, at least about 15%, at least about 20%, at least about 25%, at least about 30%, at least about 35%, at least about 40%, at least about 50%, at least about 60%, at least about 75%, at least about 80% or more. In one embodiment, a “difference of a level” may be a statistically significant difference between the specific activity of a proteasomal peptidase present in a sample as compared to a control. For example, a difference may be statistically significant if the measured level of the specific activity falls outside of about 1.0 standard deviations, about 1.5 standard deviations, about 2.0 standard deviations, or about 2.5 stand deviations of the mean of any control or reference group.

The term “enzyme linked immunosorbent assay” (ELISA) as used herein refers to an antibody-based assay in which detection of the antigen of interest is accomplished via an enzymatic reaction producing a detectable signal. ELISA can be run as a competitive or non-competitive format. ELISA also includes a 2-site or “sandwich” assay in which two antibodies to the antigen are used, one antibody to capture the antigen and one labeled with an enzyme or other detectable label to detect captured antibody-antigen complex. In a typical 2-site ELISA, the antigen has at least one epitope to which unlabeled antibody and an enzyme-linked antibody can bind with high affinity. An antigen can thus be affinity captured and detected using an enzyme-linked antibody. Typical enzymes of choice include alkaline phosphatase or horseradish peroxidase, both of which generated a detectable product upon digestion of appropriate substrates.

The term “label” as used herein, refers to any physical molecule directly or indirectly associated with a specific binding agent or antigen which provides a means for detection for that antibody or antigen. A “detectable label” as used herein refers any moiety used to achieve signal to measure the amount of complex formation between a target and a binding agent. These labels are detectable by spectroscopic, photochemical, biochemical, immunochemical, electromagnetic, radiochemical, or chemical means, such as fluorescence, chemifluorescence, or chemiluminescence, electrochemiluminescence or any other appropriate means. Suitable detectable labels include fluorescent dye molecules or fluorophores.

The term “neoplastic diseases” as used herein refers to cancers of any kind and origin and precursor stages thereof. Accordingly, the term “neoplastic disease” includes the subject matter identified by the terms “neoplasia”, “neoplasm”, “cancer”, “pre-cancer” or “tumor”. A neoplastic disease is generally manifest by abnormal cell division resulting in an abnormal level of a particular cell population. The abnormal cell division underlying a neoplastic disease is typically inherent in the cells and not a normal physiological response to infection or inflammation. In some embodiments, neoplastic diseases for diagnosis using methods provided herein include carcinoma. By “carcinoma,” it is meant a benign or malignant epithelial tumor and includes, but is not limited to, hepatocellular carcinoma, breast carcinoma, prostate carcinoma, non-small cell lung carcinoma, colon carcinoma, CNS carcinoma, melanoma, ovarian carcinoma, or renal carcinoma. An exemplary neoplastic disease includes, but is not limited to hepatocellular carcinoma.

The term “prognosis” as used herein refers to a prediction of the probable course and outcome of a clinical condition or disease. A prognosis is usually made by evaluating factors or symptoms of a disease that are indicative of a favorable or unfavorable course or outcome of the disease. The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

The terms “favorable prognosis” and “positive prognosis,” or “unfavorable prognosis” and “negative prognosis” as used herein are relative terms for the prediction of the probable course and/or likely outcome of a condition or a disease. A favorable or positive prognosis predicts a better outcome for a condition than an unfavorable or negative prognosis. In a general sense, a “favorable prognosis” is an outcome that is relatively better than many other possible prognoses that could be associated with a particular condition, whereas an unfavorable prognosis predicts an outcome that is relatively worse than many other possible prognoses that could be associated with a particular condition. Typical examples of a favorable or positive prognosis include a better than average cure rate, a lower propensity for metastasis, a longer than expected life expectancy, differentiation of a benign process from a cancerous process, and the like. For example, a positive prognosis is one where a patient has a 50% probability of being cured of a particular cancer after treatment, while the average patient with the same cancer has only a 25% probability of being cured.

As used herein, “plasma” refers to acellular fluid found in blood. Plasma may be obtained from blood by removing whole cellular material from blood by methods known in the art (e.g., centrifugation, filtration, and the like). As used herein, “peripheral blood plasma” refers to plasma obtained from peripheral blood samples.

As used herein, “scrum” includes the fraction of plasma obtained after plasma or blood is permitted to clot and the clotted fraction is removed.

The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, ubiquitinated forms, etc. Modifications also include intra-molecular crosslinking and covalent attachment to various moieties such as lipids, flavin, biotin, polyethylene glycol or derivatives thereof, etc. In addition, modifications may also include cyclization, branching and cross-linking. Further, amino acids other than the conventional twenty amino acids encoded by genes may also be included in a polypeptide.

As used herein, the term “proteasome” refers to certain large protein complexes within cells or body fluid that degrade proteins that have been tagged for elimination, particularly those tagged by ubiquitination. Proteasomes degrade denatured, misfolded, damaged, or improperly translated proteins. Proteasomal degradation of certain proteins, such as cyclins and transcription factors, serves to regulate the levels of such proteins. Exemplary proteasomes include the 26S proteasome, 20S proteasome, and the immunoproteasome.

The “26S proteasome” consists of 3 subcomplexes. The 26S proteasome consists of a 20S proteasome at the core which is capped at each end by a 19S regulatory particle (RP or PA700), The 19S RP mediates the recognition of the ubiquitinated target proteins, the ATP-dependent unfolding and the opening of the channel in the 20S proteasome, allowing entry of the target protein into the proteolytic chamber.

The “20S proteasome,” which forms the core protease (CP) of the 26S proteasome, is a barrel-shaped complex consisting of four stacked rings, each ring having 7 distinct subunits. The four rings are stacked one on top of the other and are responsible for the proteolytic activity of the proteasome. There are two identical outer a rings, having no known function, and two inner β rings, containing multiple catalytic sites. In eukaryotes, two of these sites on the β rings have chymotrypsin-like activity (Ch-L), two of these sites have trypsin-like activity (Tr-L), and two have caspase-like activity (Cas-L).

The “immunoproteasome,” which is characterized by an ability to generate major histocompatibility complex class I-binding peptides, consists of a 20S proteasome core capped on one end by 19S RP and on the other end by PA28, an activator of the 20S proteasome and an alternative RP. PA28 consists of two homologous subunits (termed α and β) and a separate but related protein termed PA28γ (also known as the Ki antigen).

The term “proteasomal peptidase activity” refers to any proteolytic enzymatic activity associated with a proteasome, such as the 26S or 20S proteasomes. The peptidase activities of proteasomes include, for example, chymotrypsin-like activity (Ch-L), trypsin-like activity (Tr-L), and caspase-like activity (Cas-L). In some embodiments, proteasomal peptidase activity is determined by measuring the rate of cleavage of a substrate per unit volume of body fluid assayed. Thus, the activity may be expressed as (moles of product formed)/time/(volume body fluid). For example, the activity may be expressed as pmol/sec/mL.

As used herein, the term “reference level” refers to a level of a substance which may be of interest for comparative purposes. In one embodiment, a reference level may be the specific activity level of a proteasomal peptidase expressed as an average of the level of the specific activity of the proteasomal peptidase from samples taken from a control population of healthy (disease-free) subjects. In another embodiment, the reference level may be the level in the same subject at a different time, e.g., before the present assay such as the level determined prior to the subject developing the disease or prior to initiating therapy. In general, samples are normalized by a common factor. For example, acellular body fluid samples are normalized by volume body fluid and cell-containing samples are normalized by protein content or cell count.

As used herein, the term “sample” may include, but is not limited to, bodily tissue or a bodily fluid such as blood (or a fraction of blood such as plasma or scrum), lymph, mucus, tears, saliva, sputum, urine, semen, stool, CSF, ascites fluid, or whole blood, and including biopsy samples of body tissue. A sample may be obtained from any subject, e.g., a subject/patient having or suspected to have a neoplastic disease.

As used herein, the term “subject” refers to a mammal, such as a human, but can also be another animal such as a domestic animal (e.g., a dog, cat, or the like), a farm animal (e.g., a cow, a sheep, a pig, a horse, or the like) or a laboratory animal (e.g., a monkey, a rat, a mouse, a rabbit, a guinea pig, or the like). The term “patient” refers to a “subject” who is, or is suspected to be, afflicted with a neoplastic disease.

As used herein, the term “specific activity” of one or more proteasomal peptidases refers to the proteasomal peptidase activity in the sample that is normalized relative to the proteasomal protein content in the sample. Specific activity of the chymotrypsin-like, trypsin-like, and caspase-like proteasomal peptidases may be designated Ch-L/p, Tr-L/p, or Cas-L/p, respectively. The skilled artisan understands that normalization of the proteasomal peptidase activity to the proteasomal protein content in the sample involves measuring and expressing the amount of proteasomal protein per unit volume of body fluid assayed, in the same type of sample (preferably a split sample) as used to measure enzymatic activity. For example, proteasomal protein may be expressed as picograms (pg) of protein per mL which, when used to normalize a proteasomal peptidase activity expressed in pmol/sec/mL, results in a specific activity expressed in pmol/sec/pg proteasomal protein.

The phrase “substantially the same as” in reference to a comparison of one value to another value for the purposes of clinical management of a disease or disorder means that the values are statistically not different. Differences between the values can vary, for example, one value may be within 20%, within 10%, or within 5% of the other value.

As used herein, the term “UPS Score” refers to a single number or score, based on a statistical analysis of the measured level of one or more biomarkers selected from the group consisting of Ch-L/p, Cas-L/p, and Tr-L/p, that reflects a relationship of a specific subject to any one particular group of individuals, such as normal individuals or individuals having a disease or any progressive state thereof. In some embodiments, the UPS score is derived from a quantitative multivariate analysis, which reflects the overall statistical assessment of an individual patient's clinical condition based upon an integrated statistical calculation of a plurality of qualitatively unique factors, e.g., specific activity of proteasomal peptidases, proteasome level, age, gender, etc.

### OVERVIEW

Disclosed herein are methods for detecting the presence or absence of neoplastic diseases in subjects based, at least in part, on results of testing methods of the present technology on a sample. Further disclosed herein are methods for monitoring the status of subjects diagnosed with neoplastic diseases based at least partially on results of tests on a sample. The test samples disclosed herein are represented by, but not limited in anyway to, sputum, blood (or a fraction of blood such as plasma, serum, or particular cell fractions), lymph, mucus, tears, saliva, urine, semen, ascites fluid, whole blood, and biopsy samples of body tissue. This disclosure is drawn, inter alia, to methods of diagnosing and monitoring neoplastic diseases using profiles of the ubiquitin-proteasome system (UPS).

The ubiquitin-proteasome system (UPS) plays a major role in the most important processes that control cell homeostasis in normal and neoplastic states. The present inventors have discovered that analyzing various components of the UPS can provide a profile that may be used for classifying and stratifying cancer patients for diagnosis, therapy, and prediction of clinical behavior.

In the context of cancer diagnosis, it is frequently difficult to have access to the diseased cells. In various embodiments, the present methods overcome problems of cancer diagnosis by determining the levels of proteasomes and proteasomal peptidase activities in the plasma of patients with neoplastic diseases. By studying the levels of proteasome, ubiquitin, and proteasome enzymatic activities in the plasma, a UPS profile of the cancer can be determined. The use of UPS profiles in diagnosing neoplastic diseases is described in further detail below and in the Examples.

In one aspect, the methods generally provide for the detection, measuring, and comparison of a pattern of UPS proteins and/or activities in a patient sample. Additional diagnostic markers may be combined with the UPS profile to construct models for predicting the presence or absence or stage of a disease. For example, clinical factors of relevance to the diagnosis of neoplastic diseases, include, but are not limited to, the patient's medical history, a physical examination, complete blood count, and other markers. Moreover, biomarkers relevant to a particular neoplastic disease may be combined with a subject's UPS profile to diagnose a disease or condition.

Neoplastic diseases to which the methods of the present invention may be applied comprise, for example, neoplastic lesions of the respiratory tract, of the urinary system, of the gastrointestinal tract of the anogenital tract, neoplastic diseases associated with HPV infection and others. Examples of cancer are cancer of the brain, breast, cervix, colon, head neck, kidney, liver, lung, non-small cell lung, melanoma, mesothelioma, ovary, sarcoma, stomach, and uterus. The term “precursor stages” in all its grammatical forms encompasses all precursor stages of cancers or any other malignancies. In particular embodiments, the methods may be applied to the diagnosis of hepatocellular carcinoma.

Accordingly, the various aspects relate to the collection, preparation, separation, identification, characterization, and comparison of the abundance of UPS proteins and/or activities in a test sample. The technology further relates to detecting and/or monitoring a sample containing one or more UPS proteins or activities, which are useful, alone or in combination, to determine the presence or absence of a neoplastic disease or any progressive state thereof.

### Sample Preparation

Test samples of acellular body fluid or cell-containing samples may be obtained from an individual or patient. Methods of obtaining test samples are well-known to those of skill in the art and include, but are not limited to, aspirations or drawing of blood or other fluids. Samples may include, but are not limited to, whole blood, serum, plasma, saliva, cerebrospinal fluid (CSF), pericardial fluid, pleural fluid, urine, and eye fluid.

In embodiments in which the proteasome activity will be determined using an acellular body fluid, the test sample may be a cell-containing liquid or an acellular body fluid (e.g., plasma or serum). In some embodiments in which the test sample contains cells, the cells may be removed from the liquid portion of the sample by methods known in the art (e.g., centrifugation) to yield acellular body fluid for the proteasome activity measurement. In suitable embodiments, serum or plasma are used as the acellular body fluid sample. Plasma and serum can be prepared from whole blood using suitable methods well-known in the art. In these embodiments, data may be normalized by volume of acellular body fluid.

In some embodiments, the proteasomal peptidase activity is determined using a cell-containing sample. In these embodiments, the cell-containing sample includes, but is not limited to, blood, urine, organ, and tissue samples. In suitable embodiments, the cell-containing sample is a blood sample, such as a blood cell lysate. Cell lysis may be accomplished by standard procedures. In certain embodiments, the cell-containing sample is a whole blood cell lysate. Kahn et al. (Biochem. Biophys. Res. Commun., 214:957-962 (1995)) and Tsubuki et al. (FEBS Lett., 344:229-233 (1994)) disclose that red blood cells contain endogenous proteinaceous inhibitors of the proteasome. However, endogenous proteasomal peptidase inhibitors can be inactivated in the presence of SDS at a concentration of about 0.05%, allowing red blood cell lysates and whole blood cell lysates to be assayed reliably. At this concentration of SDS, most if not all proteasomal peptidase activity is due to the 20S proteasome. Although purified 20S proteasome exhibits poor stability at 0.05% SDS, 20S proteasomal peptidase activity in cell lysates is stable under these conditions (Vaddi et al., U.S. Pat. No. 6,613,541).

In other embodiments, the cell-containing sample is a white blood cell lysate. Methods for obtaining white blood cells from blood are known in the art (Rickwood et al., Anal. Biochem., 123:23-31 (1982); Fotino et al., Ann. Clin. Lab. Sci., 1:131 (1971)). Commercial products useful for cell separation include without limitation Ficoll-Paque (Pharmacia Biotech) and NycoPrep (Nycomed). In some situations, white blood cell lysates provide better reproducibility of data than do whole blood cell lysates.

Variability in sample preparation of cell-containing samples can be corrected by normalizing the data by, for example, protein content or cell number. In certain embodiments, proteasomal peptidase activity in the sample may be normalized relative to the total protein content or proteasomal protein content in the sample (specific activity method). Total protein content in the sample can be determined using standard procedures, including, without limitation, Bradford assay and the Lowry method. In other embodiments, proteasomal peptidase activity in the sample may be normalized relative to cell number.

### Measuring Proteasome Level

In one embodiment, the quantity or concentration proteasomes may be measured by determining the amount of one or more proteasomal proteins in a sample. The polypeptides in the proteasome can be detected by an antibody which is detectably labeled, or which can be subsequently labeled. A variety of formats can be employed to determine whether a sample contains a proteasomal protein or proteins that bind to a given antibody. Immunoassay methods useful in the detection of proteasomal proteins include, but are not limited to, e.g., dot blotting, western blotting, protein chips, immunoprecipitation (IP), competitive and non-competitive protein binding assays, enzyme-linked immunosorbent assays (ELISA), and others commonly used and widely-described in scientific and patent literature, and many employed commercially.

Proteins from samples can be isolated using techniques that are well-known to those of skill in the art. The protein isolation methods employed can, e.g., be including, but not limited to, e.g., those described in Harlow & Lane, Antibodies: A Laboratory Manual (Cold Spring Harbor Laboratory Press, Cold Spring Harbor, N.Y., 1988). In some embodiments, proteasomal protein is extracted from the acellular body fluid sample. Plasma purification methods are known in the art such. See e.g., Cohn, E. J., et al, Am. Chem. Soc., 62:3396-3400. (1940); Cohn, E. J., et al., J. Am. Chem. Soc., 72:465-474 (1950); Pennell, R. B., Fractionation and isolation of purified components by precipitation methods, pp. 9-50. In The Plasma Proteins, Vol. 1, F. W. Putman (ed.). Academic Press, New York (1960); and U.S. Pat. No. 5,817,765.

Antibodies can be used in methods, including, but not limited to, e.g., western blots or ELISA, to detect the expressed protein complexes. In such uses, it is possible to immobilize either the antibody or proteins on a solid support. Supports or carriers include any support capable of binding an antigen or an antibody. Well-known supports or carriers include, but are not limited to, e.g., glass, polystyrene, polypropylene, polyethylene, dextran, nylon, amylases, natural and modified celluloses, polyacrylamides, gabbros, and magnetite.

Antibodies may be specific for one or more proteins that comprise the proteasomal complex. In one embodiment, the quantity or concentration of proteasomes in a sample is determined by detecting the quantity or concentration of one or more proteins that interact to form the proteasomal complex. In one embodiment, the quantity or concentration of proteasomes in a sample is determined using a polyclonal antibody to the 20S Proteasome core subunits. In other embodiments, the quantity or concentration of proteasomes in a sample is determined using a polyclonal or a monoclonal antibody directed to one or more proteins including, but not limited to, Ki-67 protein, 19S Regulator ATPase Subunit Rpt4; 19S Proteasome S5A-Subunit; 19S Proteasome S5A-Subunit; 19S Proteasome, S6-Subunit; 20S Proteasome al, 2, 3, 5, 6, & 7-Subunits; 20S Proteasome α1-Subunit; 20S Proteasome α3-Subunit; 20S Proteasome α5-Subunit; 20S Proteasome α7-Subunit; 20S Proteasome (β1-Subunit; 20S Proteasome β3-Subunit; 20S Proteasome β4-Subunit; 20S Proteasome β5i-Subunit; 26S Proteasome S4-Subunit; 26S Proteasome, S7-Subunit; Proteasome Activator PA700 Subunit 10B; 19S Regulator ATPase Subunit RptI; and 19S Regulator non-ATPase Subunit Rpn10.

Methods of generating antibodies are well known in the art, see, e.g., Sambrook, et al., 1989, Molecular Cloning: A Laboratory Manual, Second Edition, Cold Spring Harbor Press, Plainview, N.Y. Antibodies may be detectably labeled by methods known in the art. Labels include, but are not limited to, radioisotopes such as ³H, ¹⁴C, ³⁵S, ³²P, ¹²³I, ¹²⁵I, ¹³¹I), enzymes (e.g., peroxidase, alkaline phosphatase, beta-galactosidase, luciferase, alkaline phosphatase, acetylcholinesterase and glucose oxidase), enzyme substrates, luminescent substances (e.g., luminol), fluorescent substances (e.g., FITC, rhodamine, lanthanide phosphors), biotinyl groups (which can be detected by marked avidin e.g., streptavidin containing a fluorescent marker or enzymatic activity that can be detected by optical or colorimetric methods), predetermined polypeptide epitopes recognized by a secondary reporter (e.g., leucine zipper pair sequences, binding sites for secondary antibodies, metal binding domains, epitope tags) and colored substances. In binding these labeling agents to the antibody, the maleimide method (Kitagawa, T., et al., J. Biochem., 79:233-236 (1976)), the activated biotin method (Hofmann, K., et al., J. Am. Chem. Soc., 100:3585 (1978)) or the hydrophobic bond method, for instance, can be used.

In some embodiments, labels are attached via spacer arms of various lengths to reduce potential steric hindrance. Antibodies may also be coupled to electron dense substances, such as ferritin or colloidal gold, which are readily visualized by electron microscopy.

Where a radioactive label is used as a detectable substance, proteins may be localized by autoradiography. The results of autoradiography may be quantitated by determining the density of particles in the autoradiographs by various optical methods, or by counting the grains.

The antibody or sample may be immobilized on a carrier or solid support which is capable of immobilizing cells, antibodies, etc. For example, the carrier or support may be nitrocellulose, or glass, polyacrylamides, gabbros, and magnetite. The support material may have any possible configuration including spherical (e.g. bead), cylindrical (e.g. inside surface of a test tube or well, or the external surface of a rod), or flat (e.g. sheet, test strip). Indirect methods may also be employed in which the primary antigen-antibody reaction is amplified by the introduction of a second antibody, having specificity for the antibody reactive against one or more proteins that comprise a proteasome. Antibodies to proteasomal proteins are available commercially through multiple sources. For example, polyclonal antibodies directed to proteasome core subunit are available from Biomol International, Cat. No. PW8155-0100 (Plymouth, Pa.). Monoclonal antibodies directed to proteasome α subunit are available from Biomol International, Cat. No. PW8100 (Plymouth, Pa.).

Immunoassays, or assays to detect an antigen using an antibody, are well known in the art and can take many forms, e.g., radioimmunoassay, immunoprecipitation, Western blotting, enzyme-linked immunosorbent assay (ELISA), and 2-site or sandwich immunoassay.

In one embodiment, a sandwich ELISA is used. In this assay, two antibodies to different segments, or epitopes, of the antigen are used. The first antibody (capture antibody) is coupled to a solid support. When a sample of bodily fluid is contacted with the capture antibody on the solid support, the antigen contained in the bodily fluid is captured on the solid support through a specific interaction between antigen and antibody, resulting in the formation of a complex. Washing of the solid support removes unbound or non-specifically bound antigen. Subsequent exposure of the solid support to a detectably-labeled second antibody (detection antibody) to the antigen (generally to a different epitope than the capture antibody) enables the detection of bound or captured antigen. As would be readily recognized by one of skill in the art, assaying additional markers in parallel to assaying for proteasomal protein is possible with the use of distinct pairs of specific antibodies, each of which is directed against a different marker.

In an illustrative embodiment, a electro-chemiluminescent sandwich immunoassay is used. In this assay, two antibodies to different segments, or epitopes, of the antigen are used. For instance, antibody to one or more proteasomal proteins is coated on plates to capture the proteasomes. The antibody may be a mouse monoclonal antibody to proteasome alpha subunit. A sample is contacted to the plate, and after incubation under appropriate binding conditions, the plate is washed. After the wash, primary detection antibody, which binds to the one or more proteasomal proteins, is added to each well and incubated. After another wash, a Sulfo-tag labeled secondary antibody (capable of binding to the primary antibody) is added to each well and incubated for another hour. After a final wash, a MSD read buffer is added and signal is detected by MSD Sector2400 (MSD, Gaithersburg, Md.).

Relative or actual amounts of proteasomes in body fluids can be determined by methods well known in the art. See, e.g., Drach. J., et al., Cytometry, 10(6):743-749 (1989). For example, a standard curve can be obtained in the ELISA using known amounts of proteasomes, i.e., proteasome standards. The actual amount of the proteasomes in a body fluid may thus be determined using the standard curve, Another approach that does not use a standard curve is to determine the dilution of body fluid that gives a specified amount of signal. The dilution at which 50% of the signal is obtained is often used for this purpose. In this case, the dilution at 50% maximal binding of proteasomes in a patient body fluid is compared with the dilution at 50% of maximal binding for proteasomes obtained in the same assay using a reference sample (i.e., a sample taken from the corresponding bodily fluid of normal individuals, free of proliferative disorders).

Monoclonal or polyclonal antibodies may be used as the capture and detection antibodies in sandwich immunoassay systems. Monoclonal antibodies are specific for single epitope of an antigen and allow for detection and quantitation of small differences in antigen. Polyclonal antibodies can be used as the capture antibody to capture large amounts of antigen or can be used as the detection antibody. A monoclonal antibody can be used as the either the capture antibody or the detection antibody in the sandwich assay to provide greater specificity. In some embodiments, polyclonal antibodies are used as the capture antibody and monoclonal antibodies are used as the detection antibody.

One consideration in designing a sandwich ELISA is that the capture and detection antibodies should be generated against or recognize “non-overlapping” epitopes. The phrase “non-overlapping” refers to epitopes, which are segments or regions of an antigen that are recognized by an antibody, that are sufficiently separated from each other such that an antibody for each epitope can bind simultaneously. That is, the binding of one antibody (e.g., the capture antibody) to a first epitope of the antigen should not interfere with the binding of a second antibody (e.g., the detection antibody) to a second epitope of the same antigen. Capture and detection antibodies that do not interfere with one another and can bind simultaneously are suitable for use in a sandwich ELISA.

Methods for immobilizing capture antibodies on a variety of solid surfaces are well-known in the art. The solid surface may be composed of any of a variety of materials, for example, glass, quartz, silica, paper, plastic, nitrocellulose, nylon, polypropylene, polystyrene, or other polymers. The solid support may be in the form of beads, microparticles, microspheres, plates which are flat or comprise wells, shallow depressions, or grooves, microwell surfaces, slides, chromatography columns, membranes, filters, or microchips. In one embodiment, the solid support is a microwell plate in which each well comprises a distinct capture antibody to a specific marker so that multiple markers may be assayed on a single plate. In another embodiment, the solid support is in the form of a bead or microparticle. These heads may be composed of, for example, polystyrene or latex. Beads may be of a similar size or may be of varying size. Beads may be approximately 0.1 μm-10 μm in diameter or may be as large as 50 μm-100 μm in diameter.

Methods of identifying the binding of a specific binding agent to proteasomes are known in the art and vary dependent on the nature of the label. In suitable embodiments, the detectable label is a fluorescent dye. Fluorescent dyes are detected through exposure of the label to a photon of energy of one wavelength, supplied by an external source such as an incandescent lamp or laser, causing the fluorophore to be transformed into an excited state. The fluorophore then emits the absorbed energy in a longer wavelength than the excitation wavelength which can be measured as fluorescence by standard instruments containing fluorescence detectors. Exemplary fluorescence instruments include spectrofluorometers and microplate readers, fluorescence microscopes, fluorescence scanners, and flow cytometers.

In one embodiment, a sandwich assay is constructed in which the capture antibody is coupled to a solid support such as a bead or microparticle. Captured antibody-antigen complexes, subsequently bound to detection antibody, are detected using flow cytometry and is well-known in the art. Flow cytometers hydrodynamically focus a liquid suspension of particles (e.g., cells or synthetic microparticles or beads) into an essentially single-file stream of particles such that each particle can be analyzed individually. Flow cytometers are capable of measuring forward and side light scattering which correlates with the size of the particle. Thus, particles of differing sizes or fluorescent characteristics may be used in invention methods simultaneously to detect distinct markers. Fluorescence at one or more wavelengths can be measured simultaneously. Consequently, particles can be sorted by size and the fluorescence of one or more fluorescent labels can be analyzed for each particle. Exemplary flow cytometers include the Becton-Dickinson Immunocytometry Systems FACSCAN, Equivalent flow cytometers can also be used in the invention methods.

### Measuring Proteasome Activity

Proteasome activity in the test sample can be measured by any assay method suitable for determining 20S or 26S proteasome peptidase activity. (See, e.g., Vaddi et al., U.S. Pat. No. 6,613,541: McCormack et al., Biochemistry, 37:7792-7800 (1998)); Driscoll and Goldberg, J. Biol. Chem., 265:4789 (1990); Orlowski et al., Biochemistry, 32:1563 (1993)). In a suitable embodiment, a substrate having a detectable label is provided to the reaction mixture and proteolytic cleavage of the substrate is monitored by following disappearance of the substrate or appearance of a cleavage product. Detection of the label may be achieved, for example, by fluorometric, colorimetric, or radiometric assay.

Substrates for use in determining proteasomal peptidase activity may be chosen based on the selectivity of each peptidase activity. For example, the chymotrypsin-like peptidase preferentially cleaves peptides on the carboxyl side of tyrosine, tryptophan, phenylalanine, leucine, and methionine residues. The trypsin-like peptidase preferentially cleaves peptides on the carboxyl side of arginine and lysine residues. The caspase-like peptidase (or peptidylglutamyl-peptide hydrolase) preferentially cleaves peptides at glutamic acid and aspartic acid residues. Based on these selectivities, the skilled artisan can choose a specific substrates for each peptidase.

Suitable substrates for determining 26S proteasome activity include, without limitation, lysozyme, α-lactalbumin, β-lactoglobulin, insulin b-chain, and ornithine decarboxylase. When 26S proteasome activity is to be measured, the substrate is typically ubiquitinated or the reaction mixture further contains ubiquitin and ubiquitination enzymes.

In some embodiments, the substrate is a peptide less than 10 amino acids in length. In one embodiment, the peptide substrate contains a cleavable fluorescent label and release of the label is monitored by fluorometric assay. Non-limiting examples of substrates to measure trypsin-like activity include N-(N-benzoylvalylglycylarginyl)-7-amino-4-methylcoumarin (Bz-Val-Gly-Arg-AMC), N-(N-carbobenzyloxycarbonylleucylleucylarginyl)-7-amino-4-methylcoumarin (Z-Leu-Leu-Arg-AMC), Ac-Arg-Leu-Arg-AMC, and Boc-Leu-Arg-Arg-AMC. Non-limiting examples of substrates to measure caspase-like activity include N-(N-carbobenzyloxycarbonylleucylleucylglutamyl)-2-naphthylamine (Z-Leu-Leu-Glu-2NA), N—(N-carbobenzyloxycarbonylleucylleucylglutamyl)-7-amino-4-methylcoumarin (Z-Leu-Leu-Glu-AMC), and acetyl-L-glycyl-L-prolyl-L-leucyl-L-aspartyl-methylcoumarin (Ac-Gly-Pro-Leu-Asp-AMC). Non-limiting examples of substrates to measure chymotrypsin-like activity include N-(N-succinylleucylleucylvalyltyrosyl)-7-amino-4-methylcoumarin (Suc-Leu-Leu-Val-Tyr-AMC), Z-Gly-Gly-Leu-2NA, Z-Gly-Gly-Leu-AMC, and Suc-Arg-Pro-Phe-His-Leu-Leu-Val-Tyr-AMC.

Suitable substrates for measuring the chymotrypsin-like, caspase-like, and trypsin-like activities of the proteasome are Suc-Leu-Leu-Val-Tyr-AMC, Z-Leu-Leu-Glu-AMC, and Bz-Val-Gly-Arg-AMC, respectively, and the release of the cleavage product, AMC, can be monitored at 440 nm (λₑₓ=380 nm). Cleavage due to a particular peptidase may be determined by, for example, using a substrate specific for that peptidase and assaying that activity independent of other peptidases.

In certain embodiments, the reaction mixture further contains a 20S proteasome activator, Activators include those taught in Coux et al. (Ann. Rev, Biochem., 65:801-847 (1995)), such as PA2S or sodium dodecyl sulfate (SDS). However, SDS is not compatible with Bz-Val-Gly-Arg-AMC, therefore when Bz-Val-Gly-Arg-AMC is chosen as the substrate, PA28 is used instead of SDS to activate the proteasome.

### Diagnosis of Disease States

In certain embodiments, the level of one or more proteasomal peptidase activities in a test sample from a patient is used in the diagnosis of cancer. Cancer is a class of diseases characterized by uncontrolled cell division and the ability of these cells to invade other tissues, either by direct growth into adjacent tissue (invasion) or by migration of cells to distant sites (metastasis). Cancer cells may spread throughout the body (i.e., metastasize) by way of the bloodstream or lymphatic system to form tumors in other tissues or organs. Such cancers include, but are not limited to leukemia, lymphoma, breast cancer, lung cancer, esophageal cancer, stomach cancer, colorectal cancer, thyroid cancer, melanoma, bone cancer, prostate cancer, testicular cancer, ovarian cancer, cervical cancer, endometrial cancer, kidney cancer, bladder cancer, and cancer of the central nervous system.

In some embodiments, the specific activity level of one or more proteasomal peptidases (e.g., Ch-L/p, Tr-L/p, and Cas-L/p) in a test sample are used to diagnose a disease. In these embodiments, the level of proteasome activity measured in the test sample is normalized to the level of one or more proteasomal proteins to provide a specific activity value for the one or more proteasomal peptidases. The specific activity value may be compared to a reference value to determine if the levels of specific activity are elevated or reduced relative to the reference value. Typically, the reference value is the specific activity measured in a comparable sample from one or more healthy individuals. An increase or decrease in the specific activity may be used in conjunction with clinical factors other than proteasomal peptidase activity to diagnose a disease.

Association between a pathological state (e.g., a neoplastic disease) and the aberration of a specific activity level of one or more proteasomal peptidases can be readily determined by comparative analysis in a normal population and an abnormal or affected population. Thus, for example, one can study the specific activity level of one or more proteasomal peptidases in both a normal population and a population affected with a particular pathological state. The study results can be compared and analyzed by statistical means. Any detected statistically significant difference in the two populations would indicate an association. For example, if the specific activity is statistically significantly higher in the affected population than in the normal population, then it can be reasonably concluded that higher specific activity is associated with the pathological state.

Statistical methods can be used to set thresholds for determining when the specific activity level in a subject can be considered to be different than or similar to a reference level. In addition, statistics can be used to determine the validity of the difference or similarity observed between a patient's specific activity level and the reference level. Useful statistical analysis methods are described in L. D. Fisher & G. vanBelle, Biostatistics: A Methodology for the Health Sciences (Wiley-Interscience, NY, 1993). For instance, confidence (“p”) values can be calculated using an unpaired 2-tailed t test, with a difference between groups deemed significant if the p value is less than or equal to 0.05. As used herein a “confidence interval” or “CI” refers to a measure of the precision of an estimated or calculated value. The interval represents the range of values, consistent with the data that is believed to encompass the “true” value with high probability (usually 95%). The confidence interval is expressed in the same units as the estimate or calculated value. Wider intervals indicate lower precision; narrow intervals indicate greater precision. Preferred confidence intervals of the invention are 90%, 95%, 97.5%, 98%, 99%, 99.5%, 99.9% and 99.99%. A “p-value” as used herein refers to a measure of probability that a difference between groups happened by chance. For example, a difference between two groups having a p-value of 0.01 (or p=0.01) means that there is a 1 in 100 chance the result occurred by chance. Preferred p values are 0.1, 0.05, 0.025, 0.02, 0.01, 0.005, 0.001, and 0.0001. Confidence intervals and p-values can be determined by methods well-known in the art. See, e.g., Dowdy and Wearden, Statistics for Research, John Wiley & Sons, New York, 1983. Exemplary statistical tests fir associating a prognostic indicator with a predisposition to an adverse outcome are described hereinafter.

Once an association is established between a specific activity and a pathological state, then the particular physiological state can be diagnosed or detected by determining whether a patient has the particular aberration, i.e. elevated or reduced specific activity levels.

The term “elevated levels” or “higher levels” as used herein refers to levels of a specific activity that are higher than what would normally be observed in a comparable sample from control or normal subjects (i.e., a reference value). In some embodiments, “control levels” (i.e., normal levels) refer to a range of specific activity levels that would be normally be expected to be observed in a mammal that does not have a neoplastic disease. A control level may be used as a reference level for comparative purposes. “Elevated levels” refer to specific activity levels that are above the range of control levels. The ranges accepted as “elevated levels” or “control levels” are dependent on a number of factors. For example, one laboratory may routinely determine the specific activity of an enzyme in a sample that are different than the specific activity obtained for the same sample by another laboratory. Also, different assay methods may achieve different value ranges. Value ranges may also differ in various sample types, for example, different body fluids or by different treatments of the sample. One of ordinary skill in the art is capable of considering the relevant factors and establishing appropriate reference ranges for “control values” and “elevated values” of the present invention. For example, a series of samples from control subjects and subjects diagnosed with proliferative hematological disorders can be used to establish ranges that are “normal” or “control” levels and ranges that are “elevated” or “higher” than the control range.

Similarly, “reduced levels” or “lower levels” as used herein refer to levels of a peptidase specific activity that are lower than what would normally be observed in a comparable sample from control or normal subjects (i.e., a reference value). In some embodiments, “control levels” (i.e. normal levels) refer to a range of specific activity levels that would be normally be expected to be observed in a mammal that does not have a neoplastic disease and “reduced levels” refer to proteasome activity levels that are below the range of such control levels.

The specific activity level of one or more peptidases in a test sample can be used in conjunction with clinical factors other than specific activity to diagnose a disease. Clinical factors of particular relevance in the diagnosis of proliferative hematological disorders include, but are not limited to, the patient's medical history, a physical examination of the patient, complete blood count, examination of bone marrow cells, cytogenetics, and immunophenotyping of blood cells.

### Diagnosis of Hepatocellular Carcinoma

Provided herein are methods of diagnosing hepatocellular carcinoma (HCC) and differentiating HCC from chronic liver diseases. Liver cancer is the fifth most common cancer and the third leading cancer killer worldwide, and is responsible for about half million new cases and almost as many deaths per year. HCC is the major histological type of primary liver cancer. Major risk factors for developing HCC include hepatitis B virus (HBV) infection and hepatitis C virus (HCV) infection. The median survival duration is typically less than a year, because the majority of these cancers are unresectable, not suitable for new treatment modalities, and have low chemotherapy response rates. Surgical resection, such as partial hepatectomy or liver transplantation, is the most common curative treatment for the disease. However, only 20% of patients are eligible for surgery because the majority of patients are diagnosed at an advanced stage with intra- and/or extra-hepatic metastases. After curative surgery, recurrence is common and the incidence is about 50% in the first year. Thus, early detection of HCC is essential to improve survival. A diagnostic test for the detection of early-stage cancers in asymptomatic patients is one aspect of the invention.

In some embodiments, the specific activity level at one or more proteasomal peptides is combined with one or more additional HCC markers to improve diagnostic sensitivity and specificity. Exemplary HCC markers include, but are not limited to α-Fetoprotein and des-gamma carboxyprothrombin. α-Fetoprotein (AFP), a glycoprotein, is a serum biochemical marker for detection of HCC. However, AFP elevations are associated not only with HCC, but with acute and chronic liver disease as well (Lok A S, Lai C L. Hepatology 1989, 9:110-115; Yuen M F, Lai C L. Best Prac Res Clin Gastroenterol 2005, 19:91-99; Yuen M F, Lai C L. Ann Oncol 2003, 14:1463-1467; Di Bisceglie et al. J Hepatol. 2005). Thus, AFP has limited utility for detecting HCC, especially in the early stages. Recently, Lens culinaris agglutinin-reactive AFP (AFP-L3) and des-gamma-carboxyprothrombin (DCP), also known as prothrombin induced by vitamin k absence-II (PIVKA-II), have been reported to be effective in early detection of HCC. AFP-L3 is the main glycoform of AFP in the serum of HCC patients. Measurement of AFP-L3 as a percentage of total AFP helps distinguish non-malignant hepatic disease from HCC, the assessment of therapeutic effects, and predicting the prognosis of HCC (Oka et al. J Gastroenterol Hepatology. 2001; 16:1378-1383; Shiraki et al. Hepatology. 1995; 22:802-807; Miyaaki et al. J Gastroenterol. 2007; 42:962-968; Yoshida et al. International J of Oncol. 2002, 20:305-309).

DCP is an abnormal prothrombin that lack coagulating activity. It has been suggested that DCP concentration may also help to differentiate benign liver diseases and HCC. DCP has even been reported to be more sensitive and specific in diagnosing HCC when compared to AFP, especially in Eastern Asian counties and in North America, however, these results have not been shown in Europe (Ikoma et al. Hepatogastro-enterology. 2002, 49:235-238; Gomaa et al. World J Gastroenterol 2009; 15(11):1301-1314).

Multiple studies have evaluated the performance characteristics of AFP, AFPL3 and DCP in the diagnosis of HCC. These studies showed sensitivities of 77-88% and specificities of 59-91%, with differences most likely be due to the high dependency on cut-off values for each marker (Ikoma et al. Hepatogastro-enterology. 2002, 49:235-238; Sterling et al. Toyoda et al. Clinic Gastroenterol Hepatol. 2006, 4:111-117; Volk et al. Cancer Biomark. 2007, 3:79-87). These studies also showed that dependable biomarkers for early detection of HCC have remained elusive. Current diagnosis of HCC is based on imaging technology and serum AFP levels. These diagnostic tools have proven effective when the tumor burden is large (>3 cm). However, when the tumor burden is low, then these diagnostics lack the sensitivity and specificity. Consequently, most cases of HCC are diagnosed in an advanced state where treatment options are limited. Dawson has indicated the yearly fatality ratio for HCC is approaching one, and only 12% of the cases survive to 5 years post diagnosis. The present methods advantageously improve early diagnosis of HCC.

In some embodiments, a “UPS signature model” is used for the diagnosis of HCC. The model may include UPS components, proteasome and ubiquitin, proteasome enzymatic activities, Ch-L, Cas-L, Tr-L, Ch-L/P, Cas-L/P, and Tr-L/P, with gender and age, alone and in combination with conventional HCC markers, AFP, AFP-L/P, and DCP. In illustrative embodiments, the UPS signature model yields excellent diagnostic characteristics with a sensitivity of 96.5%, specificity of 99.8%, PPV of 47.6, and NPV of 99.7, respectively. However, the HCC model with 3 HCC conventional markers plus gender and age, the sensitivity, specificity, PPV, and NPV were 84.1%, 85.0%, 14.8, and 99.4, respectively. As shown in the Examples, the UPS signature model identified 35 more patients as having HCC than the HCC model when investigated in the same patient group (n=112). In some embodiments, the UPS signature model issued to diagnose patients with tumor size less than 3 cm. In some embodiments, the UPS signature model is and to differentiate patients with HCC from those with no HCC chronic liver diseases. Rather than using cutoffs from individual marker, the UPS signature model statistically weights each marker and uses the cumulative probabilities of the response categories rather than individual probability.

In one embodiment, the diagnosis of HCC is accomplished by obtaining a sample of serum from the subject and determining the level of DCP, Cas-L/p, Tr-L/p and Ch-L. In one embodiment, the method is accomplished by obtaining a sample of serum from the subject and determining the level of AFP, DCP, Cas-L/p, Tr-L/p and Ch-L. In this embodiment, intermediate value (y) is calculated as follows:

y=exp[−X+(C₁×Age)+(C₂×Gender)+(C₃×DCP)−(C₄×Ch-L)+(C₅×Cas-L/p)+(C₆×Tr-L/p)+(C₇×AFP)]

wherein X is from 16.7293 to 20.4471 inclusive;
C₁ is from 0.2027 to 0.2479 inclusive;
C₂ is from 39908 to 4.8778 inclusive;
C₃ is from 0.97557 to 1.1681 inclusive;
C₄ is from 23.5331 to 28.7627 inclusive;
C₅ is from 3.0299 to 3.7033 inclusive;
C₆ is from 0.0558 to 0.0682 inclusive;
C₇ is from 0.1534 to 0.1876 inclusive;
and wherein, age is provided in years; male gender=1, female gender=0; AFP is reported in ng/mL; DCP is reported in ng/mL; Cas-L/p is reported in pmol product/sec/pg proteasome; Tr-L/p is reported in pmol product/sec/pg proteasome; and Ch-L is reported in pmol product/sec/mL.

In a particular embodiment, the intermediate value is calculated as follows

y=exp[−18.5882+(0.2253×Age)+(4.4343×Gender)+(0.1705×AFP)+(1.0619×DCP)+(3.3666×Cas-L/p)+(0.062×Tr-L/p)−(26.1479×(Ch-L)]

wherein, age is provided in years; male gender=1, female gender=0; AFP is reported in ng/mL; DCP is reported in ng/mL; Cas-L/p is reported in pmol product/sec/pg proteasome; Tr-L/p is reported in pmol product/sec/pg proteasome; and Ch-L is reported in pmol product/sec/mL.

The intermediate value intermediate value (y) is input into a second equation to determine the end value or UPS score, wherein

UPS Score=y/(1+y)

A UPS Score greater than or equal to a cut-off value of about 0.5 is predictive of HCC in a subject. A UPS Score less than a cut-off value of about 0.5 is predictive of the absence of HCC in the subject. In certain embodiments, this cut-off value may be from 0.25 to 0.75 inclusive, or from 0.4 to 0.6 inclusive, or from 0.45 to 0.55 inclusive. Alternatively, this cut-off value may be 0.4, 0.5, or 0.6. The above numbers are subject to 5% variation.

One of skill in the art would recognize that the concentrations or activities of the markers could be provided in units other than the ones recited above. In this case, one would generate an equivalent equation to determine the intermediate value by converting the unites as recited above to other units using a mathematical function. The inverse of that function would be performed on the coefficient of that marker.

In another aspect, the invention provides is a system for diagnosing the presence of HCC in an individual. The system comprises an input device in data communication with a processor, which is in data communication with an output device.

The input device is used for entry of data including levels of AFP, DCP, Ch-L/p, Cas-L/p, Tr-L/p, Ch-L, Tr-L, and Cas-L as determined from a sample from the individual, and data for age and gender. Data may be entered manually by an operator of the system using a keyboard or keypad, Alternatively, data may be entered electronically, when the input device is a cable in data communication with a computer, a network, a server, or analytical instrument.

The processor comprises software for computing a UPS Score, and using the end value to diagnose HCC. The processor computes the UPS Score using an algorithm, wherein the algorithm is UPS Score=y/(1+y), wherein (y) is calculated as follows:

y=exp[−X+(C₁×Age)+(C₂×Gender)+(C₃×DCP)−(C₄×Ch-L)+(C₅×Cas-L/p)+(C₆×Tr-L/p)+(C₇×AFP)]

wherein X is from 16.7293 to 20.4471 inclusive:
C₁ is from 0.2027 to 0.2479 inclusive;
C₂ is from 3.9908 to 4.8778 inclusive;
C₃ is from 0.97557 to 1.1681 inclusive;
C₄ is from 23.5331 to 28.7627 inclusive;
C₅ is from 3.0299 to 3.7033 inclusive;
C₅ is from 0.0558 to 0.0682 inclusive;
C₇ is from 0.1534 to 0.1876 inclusive;
and wherein, age is provided in years; male gender=1, female gender=0; AFP is reported in ng/mL; DCP is reported in ng/mL; Cas-L/p is reported in pmol product/sec/pg proteasome; Tr-L/p is reported in pmol product/sec/pg proteasome; and Ch-L is reported in pmol product/sec/mL.

The processor further compares the UPS score to a cutoff value to diagnose the presence of HCC, wherein a UPS score greater than or equal to a cut-off value of 0.5 is predictive of HCC. A UPS score less than a cutoff value of about 0.5 is predictive of an absence of HCC. In certain embodiments, this cut-off value may be from 0.25 to 0.75 inclusive, or from 0.4 to 0.6 inclusive, or from 0.45 to 0.55 inclusive. Alternatively, this cut-off value may be 0.4, 0.5, or 0.6. The above numbers are subject to 5% variation.

The data output device, in data communication with the processor, receives the diagnosis from the processor and provides the diagnosis to the system operator. The output device can consist of for example, a video display monitor or a printer.

Monitoring Progression and/or Treatment

In one aspect, the specific activity level of one or more proteasomal peptidases (e.g., Ch-L/p, Tr-L/p, and Cas-L/p) in a biological sample of a patient is used to monitor the effectiveness of treatment or the prognosis of disease. In some embodiments, the specific activity level of one or more proteasomal peptidases in a test sample obtained from a treated patient can be compared to the level from a reference sample obtained from that patient prior to initiation of a treatment. Clinical monitoring of treatment typically entails that each patient serve as his or her own baseline control. In some embodiments, test samples are obtained at multiple time points following administration of the treatment. In these embodiments, measurement of specific activity level of one or more proteasomal peptidases in the test samples provides an indication of the extent and duration of in vivo effect of the treatment,

### Determining Prognosis

A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example, prognosis can be expressed as a percent chance that a patient will survive after one year, five years, ten years or the like. Alternatively, prognosis may be expressed as the number of years, on average that a patient can expect to survive as a result of a condition or disease. The prognosis of a patient may be considered as an expression of relativism, with many factors affecting the ultimate outcome. For example, for patients with certain conditions, prognosis can be appropriately expressed as the likelihood that a condition may be treatable or curable, or the likelihood that a disease will go into remission, whereas for patients with more severe conditions prognosis may be more appropriately expressed as likelihood of survival for a specified period of time.

Additionally, a change in a clinical factor from a baseline level may impact a patient's prognosis, and the degree of change in level of the clinical factor may be related to the severity of adverse events. Statistical significance is often determined by comparing two or more populations, and determining a confidence interval and/or a p value.

Multiple determinations of proteasomal specific activity levels can be made, and a temporal change in activity can be used to determine a prognosis. For example, comparative measurements are made of the specific activity of an acellular body fluid in a patient at multiple time points, and a comparison of a specific activity value at two or more time points may be indicative of a particular prognosis.

A prognosis is often determined by examining one or more clinical factors and/or symptoms that correlate to patient outcomes. As described herein, the specific activity level of a proteasomal peptidase is a clinical factor useful in determining prognosis. The skilled artisan will understand that associating a clinical factor with a predisposition to an adverse outcome may involve statistical analysis.

In certain embodiments, the levels of specific activity of one or more proteasomal peptidases are used as indicators of an unfavorable prognosis. According to the method, the determination of prognosis can be performed by comparing the measured specific activity level to levels determined in comparable samples from healthy individuals or to levels known to corresponding with favorable or unfavorable outcomes. The absolute specific activity levels obtained may depend on an number of factors, including, but not limited to, the laboratory performing the assays, the assay methods used, the type of body fluid sample used and the type of disease a patient is afflicted with. According to the method, values can be collected from a series of patients with a particular disorder to determine appropriate reference ranges of specific activity for that disorder. One of ordinary skill in the art is capable of performing a retrospective study that compares the determined specific activity levels to the observed outcome of the patients and establishing ranges of levels that can be used to designate the prognosis of the patients with a particular disorder. For example, specific activity levels in the lowest range would be indicative of a more favorable prognosis, while specific activity levels in the highest ranges would be indicative of an unfavorable prognosis. Thus, in this aspect the term “elevated levels” refers to levels of specific activity that are above the range of the reference value. In some embodiments patients with “high” or “elevated” specific activity levels have levels that are higher than the median activity in a population of patients with that disease. In certain embodiments, “high” or “elevated” specific activity levels for a patient with a particular disease refers to levels that are above the median values for patients with that disorder and are in the upper 40% of patients with the disorder, or to levels that are in the upper 20% of patients with the disorder, or to levels that are in the upper 10% of patients with the disorder, or to levels that are in the upper 5% of patients with the disorder.

Because the level of specific activity in a test sample from a patient relates to the prognosis of a patient in a continuous fashion, the determination of prognosis can be performed using statistical analyses to relate the determined specific activity levels to the prognosis of the patient. A skilled artisan is capable of designing appropriate statistical methods. For example, the methods may employ the chi-squared test, the Kaplan-Meier method, the log-rank test, multivariate logistic regression analysis, Cox's proportional-hazard model and the like in determining the prognosis. Computers and computer software programs may be used in organizing data and performing statistical analyses.

In certain embodiments, the prognosis of cancer patients can be correlated to the clinical outcome of the disease using the specific activity level and other clinical factors. Simple algorithms have been described and are readily adapted to this end. The approach by Giles et, al., British Journal of Hemotology, 121:578-585, is exemplary. As in Giles et al., associations between categorical variables (e.g., proteasome activity levels and clinical characteristics) can be assessed via crosstabulation and Fisher's exact test. Unadjusted survival probabilities can be estimated using the method of Kaplan and Meier. The Cox proportional hazards regression model also can be used to assess the ability of patient characteristics (such as proteasome activity levels) to predict survival, with ‘goodness of fit’ assessed by the Grambsch-Therneau test, Schoenfeld residual plots, martingale residual plots and likelihood ratio statistics (see Grambsch et al, 1995). In some embodiments, this approach can be adapted as a simple computer program that can be used with personal computers or personal digital assistants (PDA). The prediction of patients' survival time in based on their proteasome activity levels can be performed via the use of a visual basic for applications (VBA) computer program developed within Microsoft Excel. The core construction and analysis may be based on the Cox proportional hazard models. The VBA application can be developed by obtaining a base hazard rate and parameter estimates. These statistical analyses can be performed using a statistical program such as the SAS® proportional hazards regression, PHREG, procedure. Estimates can then be used to obtain probabilities of surviving from one to 24 months given the patient's covariates. The program can make use of estimated probabilities to create a graphical representation of a given patient's predicted survival curve. In certain embodiments, the program also provides 6-month, 1-year and 18-month survival probabilities. A graphical interface can be used to input patient characteristics in a user-friendly manner.

In some embodiments of the invention, multiple prognostic factors, including specific activity level, are considered when determining the prognosis of a patient. For example, the prognosis of a cancer patient may be determined based on specific activity and one or more prognostic factors selected from the group consisting of cytogenetics, performance status, age, gender and previous diagnosis. In certain embodiments, other prognostic factors may be combined with the specific activity level or other biomarkers in the algorithm to determine prognosis with greater accuracy.

### Kits

A kit may be used for conducting the diagnostic and prognostic methods described herein. Typically, the kit should contain, in a carrier or compartmentalized container, reagents useful in any of the above-described embodiments of the diagnostic method. The carrier can be a container or support, in the form of, e.g., bag, box, tube, rack, and is optionally compartmentalized. The carrier may define an enclosed confinement for safety purposes during shipment and storage. In one embodiment, the kit includes an antibody selectively immunoreactive with a proteasome. The antibodies may be labeled with a detectable marker such as radioactive isotopes, or enzymatic or fluorescence markers. Alternatively, secondary antibodies such as labeled anti-IgG and the like may be included for detection purposes. In addition, reagents to detect the activity of one or more proteasomal peptidases may be provided. Optionally, the kit can include standard proteasomes prepared or purified for comparison purposes. Instructions for using the kit or reagents contained therein are also included in the kit.

### Example

The present methods and kits, thus generally described, will be understood more readily by reference to the following examples, which are provided by way of illustration and are not intended to be limiting of the present methods and kits. The following is a description of the materials and experimental procedures used in the example.

### Materials and Methods

Study subjects. A total of 312 subjects were studied. The first group consisted of 112 patients with HCC. The diagnosis of HCC was confirmed by (1) histology; (2) new hepatic lesions with an AFP of >1,000 ng/mL; or (3) new hepatic lesions with arterial phase enhancement on computerized tomography (CT) or magnetic resonance imaging (MRI). The second group included 60 patients with HCV-related CLD and not HCC. HCV infection was detected by polymerase chain reaction analysis. The CLD group had at least 2 years of follow-up with no evidence of development of HCC. All HCC and CLD samples were obtained from the Liver Center, Harvard Medical School, Boston, Mass., and were stored at −80° C. A third group of 140 apparently healthy adults with no known hepatitis or liver diseases was recruited from the Clinical Correlation Department at Quest Diagnostics Nichols Institute, San Juan Capistrano, Calif., All samples were collected with an IRB-approved protocol and consent form. The serum samples were isolated from peripheral blood and stored at −80° C. until analysis.

Measurement of total AFP, AFP-L3 and DCP. Total AFP, AFP-L3% and DCP serum levels were measured using two commercially available kits in the LiBASys automated immunological analyzer (Wako Chemicals USA Inc. Richmond, Va.). AFP and AFP-L3 were measured simultaneously using a liquid-phase binding reaction between antigen and antibody, and separation of bound and free forms by anion exchange column chromatography. The cutoff value of AFP for HCC was set at 20 ng mL, the most commonly used clinical cutoff value. The cutoff value of AFP-L3% for HCC was set at 10%, as indicated by the kit manufacturer and verified by our laboratory. The DCP assay is based on anti-DCP monoclonal antibodies and anti-prothrombin monoclonal antibodies, and a substrate for fluorophotometric measurement. The cutoff for DCP for HCC was 7.5 ng/ml as indicated by kit manufacturer and verified by our laboratory. In some specimens tested, the AFP, AFP-L3 or DCP results were reported as “not detectable” or “not reportable” due to low AFP, AFP-L3 or DCP levels in the sample. For the purpose of quantitative analysis, all samples with “undetectable” or “not reportable” results were considered to have values of 0.1 ng/ml for AFP, 0.1% for AFP-L3, and 0.1 ng/mL for DCP.

Measurement of proteasome level. Proteasome levels were measured using an immunoassay based on electro-chemiluminescence technology (MesoScale Discovery, Gaithersburg, Md.). A monoclonal antibody (MCP20, Biomol International, Cat. No. PW8100, Plymouth, Pa.) specific to proteasome alpha subunit was captured on a MSD goat anti-mouse plate. Proteasome standards (Biomol International, Cat. No. PW8720, Plymouth, Pa.), control and patient serum samples (1:20 dilution in MSD lyses buffer) were added to the wells and incubated at room temperature (RT) for 2 h. After washing, the detection antibody (Biomol International, Cat. No, PW8155-0100, Plymouth, Pa.), a rabbit polyclonal antibody against the proteasome core subunit, was added to the well and incubated at RT for 1 h. The plate was washed and incubated with sulfo-tag-labeled goat anti-rabbit antibody at RT for 1 h. Following the final wash, MSD read buffer was added to each well, and signal was detected on a MSD SECTOR™ Imager (MSD, Gaithersburg, Md.). The proteasome level in human serum (ng/mL) was calculated using proteasome standard curve. Sensitivity of the proteasome MSD assay was 100 pg/mL.

Measurement of circulating ubiquitin level. The level of ubiquitin in serum was detected by an immunoassay using electro-chemiluminescence-based technology. Briefly, a MSD plate was blocked with goat anti-mouse antibodies for 2 hours. Then, an anti-ubiquitin monoclonal antibody (clone FK1, Cat, No. PW8805, Biomol International, Plymouth, Pa.) was coated on the MSD goat anti-mouse plate at 4° C. on a shaker for overnight. HeLa cell lysate was used for standards, and ubiquitin positive (Catalog No. 89899, Pierce, Rockford, Ill.) and negative controls were used in the assay. Serum samples were diluted 1:2 using the MSD lysis buffer. Controls, standards and serum samples were added to the wells and incubated at RT for 3 h on a shaken During incubation, any ubiquitin present in samples or standards was specifically captured by the anti-ubiquitin. After washing, sulfo-tag-labeled anti-ubiquitin antibody was added to each well and incubated at RT for 1 h. After the final wash, MSD read buffer was added to the wells and signal was detected on an MSD SECTOR™ Imager (MSD, Gaithersburg, Md.). The ubiquitin levels (ng/mL) were extrapolated from reference standard curve. The sensitivity of the assay was 2 ng/mL.

Measurement of circulating proteasomal peptidase activities. The measurement of proteasome enzymatic activities has been previously described (Ma et al. Cancer. 2008, 112(6):1306-12). Briefly, chymotrypsin-like (Ch-L), caspase-like (Cas-L), and trypsin-like (Tr-L) activities were assayed by continuously monitoring the production of 7-amino-4-methylcoumarin (AMC) from fluorogenic peptides. The release of free AMC was measured on a SpectraMax Gemini EM instrument (Molecular Devices Corporation, Sunnyvale, Calif.) with the following parameters: excitation, 380 nm; emission, 460 nm; read interval, 1 min; read length, 30 min; temperature, 37° C. Enzymatic activities were quantitated by generating a standard curve of AMC (range, 0-8 μM). The slope of the AMC standard curve was then used as a conversion factor to calculate the activity of each individual sample as pmol AMC/second/mL serum. The specific activity of each proteasomal peptidase (Ch-L/p, Tr-L/p, and Cas-L/p) was also normalized to the amount of proteasomes in the sample and expressed as pmol AMC/sec/pg proteasome.

Determination of specific enzymatic activities of proteasomes. To determine the specific enzymatic activities of proteasomes, the level of the enzymatic activity was divided by the level of proteasome protein in the same quantity of serum sample. Therefore, three new values were generated: Ch-L specific activity (Ch-L/p)=Ch-L/proteasome level; Cas-L specific activity (Cas-L/p)=Cas-L/proteasome level; and Tr-L specific activity (Tr-L/p)=Tr-L/proteasome level.

Statistical analysis. Relationships between HCC and 11 biochemical markers, gender and age were investigated using logistic regression analysis. First, univariate logistic regression was used to assess the association of HCC with each of the biomarkers, age, and gender. Second, all biomarkers combined with age and gender were analyzed as independent variables by multivariate logistic regression analysis to predict HCC. The models with different combinations of markers were compared using area under the receiver operating characteristic (AUROC) curve analysis. A single model—called the UPS signature model—was selected based on the fewest variables yielding the most favorable AUROC. For comparison purposes, a separate model was created using only HCC markers, AFP, AFP-L3, DCP, gender and age, named HCC model. The sensitivity, specificity, positive predictive value (PPV) and negative predictive value (NPV) were calculated using various cutoff points ranging from 0.0 to 1.0. A final cutoff score of 0.5 was used to predict presence (<0.5) or absence of HCC (≧0.5). The UPS signature model used in the example is as follows:

y=exp[−18.5882+(0.2253×Age)+(4.4343×Gender)+(0.1705×AFP)+(1.0619×DCP)+(3.3666×Cas-L/p)+(0.062×Tr-L/p)−(26.1479×Ch-L)]

UPS score=y/(1+y)

Clinical and demographic characteristics of HCC, CLD and normal donor groups were compared by Student's t test for continuous variables and Fisher's exact test for categorical variables. The 95% confidence intervals (CIs) were computed for sensitivity and specificity using binomial distribution. PPV and NPV were calculated based on sensitivity and specificity with prevalence of 3% using formulas from Altman. All statistical analyses were performed using SAS 9.1.3 software (SAS, Cary, N.C.).

### Study Population

The demographic and clinical characteristics of the HCC, CLD, and apparently healthy groups are summarized in Table 1. All patients with HCC had underlying cirrhosis as determined by biopsy. Among patients with HCC, there were 20.5% (n=23) with HBV, 58.9% (n=66) with HCV, 1.8% (n=2) with HBV and HCV, and 18.8% (n=21) associated with alcohol or Nonalcoholic Steatohepatitis. Forty-four (39.3%) patients had small tumors (≦3 cm, range 0.2-3 cm) and sixty-eight (60.7%) had large tumor (>3 cm, range 3-6 cm). Ninety one patients (81.3%) had one tumor and twenty one (18.7%) had multiple tumors. Sixty cirrhotic patients had confirmed HCV infection as determined by serum HCV-RNA polymerase chain reaction analysis. The normal population group was comprised of 140 healthy individual with 50 (35.7%) men, and 90 (64.3%) women.

TABLE 1
Clinical Characteristics of Study Subjects

| | | Chronic Liver | Normal |
|---|---|---|---|
| | HCC | Diseases | Population |
| Number | 112 | 60 | 140 |
| Age, Median (Range) | 56 (25-80) | 51 (19-72) | 34 (18-61) |
| Male n (%) | 97 (86.6%) | 39 (65.0%) | 50 (35.7%) |
| Etiology n (%) | | | |
| HBV | 23 (20.5%) | 0 | NA |
| HCV | 66 (58.9%) | 60 (100%) | NA |
| HBV, HCV | 2 (1.8%) | 0 | NA |
| Others* | 21 (18.8%) | 0 | NA |
| Metavir, n (%) | | | |
| 0 | NA | 7 (12%) | NA |
| 1 | NA | 21 (35%) | NA |
| 2 | NA | 2 (3%) | NA |
| 3 | NA | 2 (3%) | NA |
| 4 | NA | 7 (12%) | NA |
| Tumor Size, n (%) | | | |
| ≦3 cm | 44 (39.3%) | NA | NA |
| >3 cm | 68 (60.7%) | NA | NA |
| Tumor number, n (%) | | NA | NA |
| 1 | 91 (81.3%) | NA | NA |
| 2 | 5 (4.5%) | NA | NA |
| 3 | 3 (2.7%) | NA | NA |
| 4 | 13 (11.6%) | NA | NA |

Abbreviations:
HCC, hepatocellular carcinoma;
NA, not applicable
*Others includes alcohol and nonalcoholic steatohepatitis

### Levels of Biomarkers in Normal, HCC, and Chronic Liver Disease Patients.

The results of all markers analyzed in the HCC, CLD and control groups are shown in Table 2. Median serum levels of AFP, AFP-L3, DCP, Tr-L, and Ch-L/p were significantly higher in the HCC group than in CLD patients, whereas median ubiquitin levels were significantly lower in patients with HCC. Levels of proteasome, Ch-L, Cas-L, Tr-L/p and Cas-L/p did not differ significantly between these two groups. There were significant differences in the levels of all markers, except Cas-L/p, between patients with HCC and the normal control group. Similarly, serum levels of all UPS components and their enzymatic activities were significantly different in patients with CLD than in the control group. In univariate logistic regression analysis, levels of AFP, AFP-L3, DCP, ubiquitin, Tr-L, and Cas-L/p, as well as age and gender, were significantly associated with the risk of HCC (Table 3).

TABLE 2
Median Levels of UPS biomarkers, AFP, AFP-L3, and DCP in Study Patients

| | HCC | | Chronic Liver Diseases | | Normal | | P value* | P value* | P value* |
|---|---|---|---|---|---|---|---|---|---|
| | Median (range) | | Median (range) | | Median (range) | | HCC vs. | HCC vs. | CLD vs. |
| Marker | n = 112 | | n = 60 | | n = 140 | | CLD | Normal | Normal |
| AFP | 40.8 | (0.1-293100.0) | 1.8 | (0.1-74.3) | 2.7 | (0.1-8.8) | 0.0016 | 0.0016 | 0.0369 |
| (ng/mL) | | | | | | | | | |
| AFP-L3% | 0.1 | (0.1-96.7) | 0.1 | (0.1-16.5) | 0.1 | (0.1-0.1) | <0.0001 | <0.0001 | 0.3214 |
| (% AFP-L3 of | | | | | | | | | |
| total AFP) | | | | | | | | | |
| DCP | 6.8 | (0.1-4790.0) | 0.1 | (0.1-6.3) | 0.1 | (0.1-3.8) | 0.0011 | 0.0011 | 0.6774 |
| (ng/mL) | | | | | | | | | |
| Proteasome | 369.33 | (97.7-2975.0) | 498.03 | (200.01-2679.30) | 235.15 | (47.14-3540.90) | 0.6396 | <0.0001 | <0.0001 |
| (pg/mL) | | | | | | | | | |
| Ubiquitin | 68.57 | (8.42-185.40) | 89.15 | (33.46-506.94) | 53.86 | (8.06-160.46) | 0.0009 | 0.0004 | <0.0001 |
| (pg/mL) | | | | | | | | | |
| Ch-L | 0.40 | (0.05-4.43) | 0.43 | (0.09-1.59) | 0.35 | (0.11-1.15) | 0.2295 | 0.0037 | 0.003 |
| (pmol AMC/sec/mL) | | | | | | | | | |
| Tr-L | 4.84 | (0.22-26.51) | 7.23 | (3.05-19.80) | 11.36 | (2.18-27.44) | 0.0001 | <0.0001 | <0.0001 |
| (pmol AMC/sec/mL) | | | | | | | | | |
| Cas-L | 0.95 | (0.14-10.17) | 0.93 | (0.30-3.97) | 0.67 | (0.16-2.73) | 0.0284 | <0.0001 | 0.0004 |
| (pmol AMC/sec/mL) | | | | | | | | | |
| Ch-L/p | 0.99 | (0-3.44) | 0.85 | (0.19-2.70) | 1.37 | (0.08-4.51) | 0.0086 | 0.0001 | <0.0001 |
| (pmol AMC/sec/pg | | | | | | | | | |
| proteasome) | | | | | | | | | |
| Tr-L/p | 11.66 | (0-116.59) | 14.41 | (4.46-58.97) | 42.5 | (1.78-270.58) | 0.6315 | <0.0001 | <0.0001 |
| (pmol AMC/sec/pg | | | | | | | | | |
| proteasome) | | | | | | | | | |
| Cas-L/p | 2.58 | (0-59.01) | 1.99 | (0.58-5.36) | 3.05 | (0.15-7.39) | 0.0285 | 0.7751 | <0.0001 |
| (pmol AMC/sec/pg | | | | | | | | | |
| proteasome) | | | | | | | | | |

Abbreviation: AFP, alpha fetoprotein; DCP, des-gamma-carboxyprothrombin; Tr-L, trypsin-like; Tr-L/p, trypsin-like specific activity; Ch-L, chymotrypsin-like; Ch-L/p, chymotrypsin-like specific activity; Cas-L, caspase-like,; cas-L/p, caspase-like specific activity;
*P values were calculated by Student's t test.

TABLE 3
Univariate Logistic Regression Analysis for Differentiating HCC from Chronic Liver Diseases

| Variable | Coefficient | Coefficient SE | Chi-Square | Coefficient P* | OR (95% CI) |
|---|---|---|---|---|---|
| AFP | 0.0410 | 0.0129 | 58.28 | <0.0001 | 1.0419 (1.0158:1.0686) |
| DCP | 0.6613 | 0.1861 | 79.40 | <0.0001 | 1.9373 (1.3453:2.7898) |
| AFP-L3 | 0.2181 | 0.0933 | 42.72 | <0.0001 | 1.2437 (1.0358:1.4934) |
| Ubiquitin | −0.0190 | 0.0050 | 18.77 | <0.0001 | 0.9812 (0.9716:0.9909) |
| Tr-L | −0.1613 | 0.0468 | 14.28 | 0.0002 | 0.8510 (0.7764:0.9329) |
| Tr-L/p | 0.0037 | 0.0091 | 0.16 | 0.6854 | 1.0037 (0.9859:1.0218) |
| Ch-L | 0.3263 | 0.3496 | 1.01 | 0.3144 | 1.3858 (0.6984:2.7495) |
| Ch-L/p | 0.5577 | 0.2595 | 5.10 | 0.0239 | 1.7466 (1.0502:2.9048) |
| Cas-L | 0.2317 | 0.1400 | 3.59 | 0.0580 | 1.2607 (0.9581:1.6589) |
| Cas-L/p | 0.3596 | 0.1281 | 9.95 | 0.0016 | 1.4328 (1.1145:1.8419) |
| Proteasome | 0.0001 | 0.0003 | 0.17 | 0.6763 | 1.0001 (0.9995:1.0008) |
| Age | 0.0764 | 0.0195 | 19.84 | <0.0001 | 1.0794 (1.0389:1.1214) |
| Gender | 1.2579 | 0.3875 | 10.76 | 0.0010 | 3.5179 (1.6462:7.5180) |

Abbreviations: AFP, alpha-fetoprotein; DCP, des-gamma-carboxyprothrombin; Tr-L, trypsin-like; Tr-L/p, trypsin-like specific activity; Ch-L, chymotrypsin-like; Ch-L/p, chymotrypsin-like specific activity; Cas-L, caspase-like; cas-L/p, caspase-like specific activity; OR, odds ratio.
*P values were calculated by Student's t test or Fisher's exact test.

TABLE 4
Multivariate Logistic Regression Model for Differentiating
HCC from CLD

| | | Coefficient | Coefficient |
|---|---|---|---|
| Variable | Coefficient | SE | P |
| Age | 0.2253 | 0.0854 | 0.0006 |
| Sex | 4.4343 | 1.6778 | 0.0006 |
| AFP | 0.1705 | 0.0459 | <0.0001 |
| DCP | 1.0619 | 0.6061 | <0.0001 |
| Cas-L/p | 3.3666 | 1.0035 | <0.0001 |
| Tr-L/p | 0.0620 | 0.0451 | 0.1025 |
| Ch-L | −26.1500 | 7.5580 | <0.0001 |

Abbreviations:
AFP, alpha-fetoprotein;
DCP, des-gamma-carboxy prothrombin;
Tr-L/p, trypsin-like specific activity;
Ch-L, chymotrypsin-like;
Cas-L/p, caspase-like specific activity.

Model for Differentiating HCC from CLD

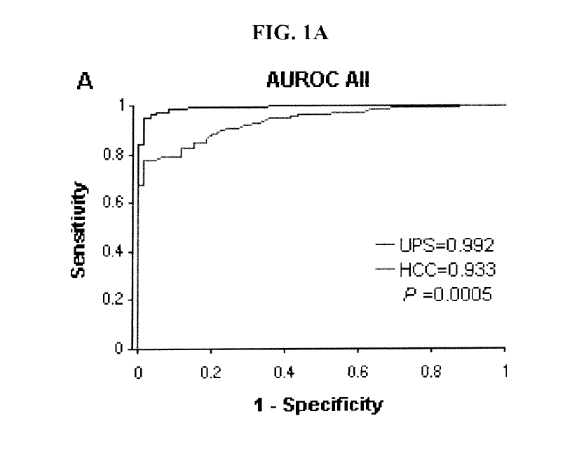

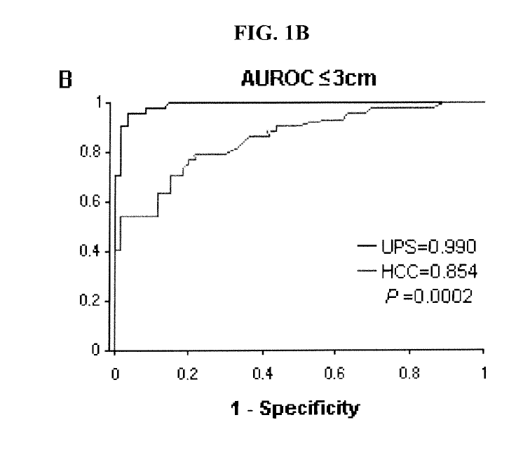

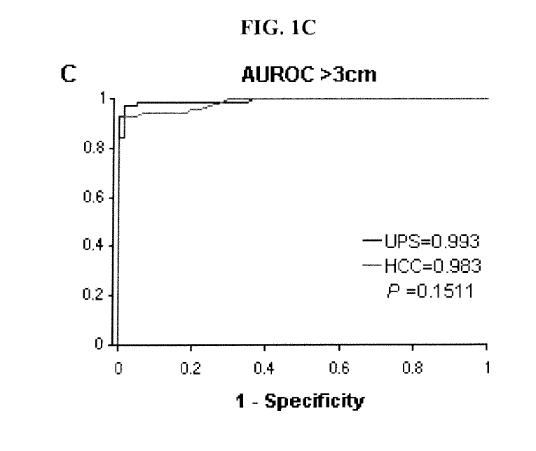

Factors that were independently associated with HCC in multivariate analysis were evaluated in various combinations to determine if additional diagnostic power could be achieved by combining UPS markers with the conventional HCC markers AFP, AFP-L3, and DCP; age and gender were also combined with all markers. The optimal multivariate model giving the largest AUROC consisted of Ch-L, Tr-L/p, Cas-L/p, AFP, DCP, age, and gender (Table 4). This UPS signature model yielded an AUROC of 0.992 (95% CI, 0.983-1.000), significantly greater than that of the HCC marker model that included AFP, AFP-L3, DCP, gender, and age [(AUROC=0.933; 95% CI, 0.899-0.968) (P=0.0005) (FIG. 1a)]. The greater discriminatory ability of the UPS signature appeared to be largely due to better performance in patients with small (≦3 cm) tumors [(AUROC=0.990 vs. 0.854) (P=0.0002) (FIG. 1b)]. Whereas the UPS signature and HCC marker models had very similar AUROC values when analysis was limited to patients with large tumors [(AUROC=0.993 vs. 0.983) (FIG. 1c)].

Accuracy of the UPS Signature Model for Differentiating HCC from CLD

The diagnostic accuracy of the UPS signature model for differentiating HCC from CLD is presented in Table 5. A cutoff score of 0.5 was used to predict HCC: values ≧0.5 indicate a high probability of HCC, and values <0.5 indicate a low probability of HCC. Among patients with HCC, 108 (96.4%) of 112 had a score ≧0.5, suggesting the presence of HCC. Among the 60 CLD patients with no HCC, 58 (96.7%) had a score <0.5 and thus would have been interpreted as having a low likelihood of HCC. When compared with the HCC marker model, the UPS signature model resulted in significantly improved sensitivity (P=0.0005) and PPV (P=0.029). The UPS signature model also showed dramatic improvement over the 3 conventional HCC markers (Table 5). When analysis of HCC patients was restricted to those with small tumors (≦3 μm), the UPS signature model still yielded significantly greater sensitivity, specificity, and PPV than the HCC marker model and the 3 conventional HCC markers. For HCC patients with large tumors (>3 cm), the UPS signature model yielded a PPV of 47.4 compared with 16.3 for the HCC marker model. There were no significant differences in the sensitivity between the UPS signature model and HCC marker model, or between the UPS model and 3 HCC markers with cutoff (P>0.05, Table 5).

TABLE 5
Multivariate Logistic Regression Model for Differentiating HCC
from Chronic Liver Diseases

| | | | |
|---|---|---|---|
| Sensitivity % | Specificity % | PPV % | NPV % |

Total patients with HCC (n = 112)

| | | | | |
|---|---|---|---|---|
| AFP, AFP-L3, DCP* | 76.1 | 88.3 | 16.8 | 99.2 |
| HCC Marker Model | 84.1 | 85.0 | 14.8 | 99.4 |
| UPS Signature Model | 96.4 | 96.7 | 47.2 | 99.9 |

Patients with small tumor (≦3 cm, n = 44)

| | | | | |
|---|---|---|---|---|
| AFP, AFP-L3, DCP* | 54.5 | 88.3 | 12.6 | 98.9 |
| HCC Marker Model | 68.2 | 85.0 | 12.3 | 99.9 |
| UPS Signature Model | 95.5 | 96.7 | 47.0 | 99.7 |

Patients with large tumor (>3 cm, n = 68)

| | | | | |
|---|---|---|---|---|
| AFP, AFP-L3, DCP* | 89.9 | 88.3 | 19.2 | 99.6 |
| HCC Marker Model | 94.2 | 85.0 | 16.3 | 99.8 |
| UPS Signature Model | 97.1 | 96.7 | 47.4 | 99.9 |

Abbreviations:
AFP, alpha fetoprotein;
DCP, des-gamma-carboxyprothrombin;
PPV, positive predictive value;
NPV, negative predictive value.
*The following cutoffs were used to calculate sensitivity, specificity, PPV, and NPV: AFP, 20 ng/mL, APF-L3%, 10%; and DCP, 7.5 ng/mL. An HCC prevalence of 3% was assumed for PPV and NPV calculations.

Taken together, these data demonstrate the utility of UPS components and their enzymatic activities, alone and in combination with conventional HCC markers to improve HCC detection. The data demonstrate that the UPS signature model, a multivariate logistic regression model, composed of Ch-L, Tr-L/p, Cas-L/p, AFP, DCP, gender, and age yielded superior sensitivity for detecting HCC than each marker alone, or compound with numerous alternative models. The UPS signature model increased PPV from 16.8 to 47.2, when compared with HCC model.

The performance characteristics of UPS components and their enzymatic activities were compared with HCC conventional markers and a statistic model, the UPS signature model, was devised which can categorize patients into HCC and no HCC chronic liver diseases. The results showed that multivariate logistic regression model had excellent performance for the diagnosis of HCC. The multivariate model was also able to detect HCC patients with small tumors. This has important implications for its utility in screening and early detection of HCC. Finally, this is the first model for HCC detection that utilizes age and gender in a multivariate analysis.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The inventions illustratively described herein may suitably be practiced in the absence of any element or elements, limitation or limitations, not specifically disclosed herein. Thus, for example, the teens “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A method for diagnosing a neoplastic disease in a subject, the method comprising: determining, in an acellular body fluid sample from the subject, the specific activity of one or more proteasomal peptidases selected from the group consisting of chymotrypsin-like activity (Ch-L), trypsin-like activity (Tr-L), and caspase-like activity (Cas-L), wherein the specific activity is determined by normalizing the one or more peptidase activities to the amount of proteasomal protein in the sample, and diagnosing the subject as having a neoplastic disease when a difference in the specific activity of one or more proteasomal peptidases compared to a reference specific activity indicates a neoplastic disease in the subject.

2. The method of claim 1 wherein the neoplastic disease is hepatocellular carcinoma.

3. The method of claim 1, wherein the acellular body fluid is selected from the group consisting of scrum and plasma.

4. The method of claim 1, wherein the reference specific activity is the specific activity in a comparable sample from one or more healthy individuals.

5. The method of claim 1, wherein the level of specific activity of one or more proteasomal peptidases is compared to a cutoff value determined from the level of specific activity of one or more proteasomal peptidases present in a comparable sample from healthy individuals, and wherein an increase or decrease in the subject value relative to the cutoff value is used to determine a diagnosis for the subject.

6. A method of diagnosing a neoplastic disease in a subject, the method comprising:
determining the amount of proteasomal protein in a test sample for the subject;
determining the level of one or more proteasomal peptidase activities in a test sample from the subject, the peptidase activities selected from the group consisting of chymotrypsin-like activity (Ch-L), trypsin-like activity (r-L), and caspase-like activity (Cas-L),
normalizing the level of one or more proteasomal peptidase activities to the amount of proteasomal protein to provide a specific activity of the one or more proteasomal peptidases; and
using the specific activity of the one or more proteasomal peptidases to diagnose the presence of a neoplastic disease in the subject.

7. The method of claim 6, wherein the neoplastic disease is hepatocellular carcinoma.

8. The method of claim 6, wherein the test sample is an acellular body fluid sample.

9. The method of claim 8, wherein the acellular body fluid is selected from the group consisting of serum and plasma.

10. The method of claim 6, wherein the test sample is a cell-containing sample.

11. The method of claim 6, wherein the specific activity of one or more proteasomal peptidases is compared to a cutoff value determined from the specific activity of one or more proteasomal peptidases present in a comparable sample from healthy individuals, and wherein an increase or decrease in the subject specific activity relative to the cutoff value is used to determine a prognosis for the subject.

12. A method for diagnosing hepatocellular carcinoma (HCC) in a subject, the method comprising:
(a) assaying the amount of one or more of chymotrypsin-like activity (Ch-L), trypsin-like activity (Tr-L), and caspase-like activity (Cas-L) in a sample from the subject;
(b) assaying the amount of one or more of alpha-fetoprotein (AFP), AFP-L3, des-gamma-carboxyprothrombin (DCP), and ubiquitin in the sample
(c) assaying the amount of proteasomal protein in the sample and normalizing one or more of Ch-L, Tr-L, and Cas-L to determine the specific activity;
(d) determining a score for the subject based on the results obtained in steps (a) and (b); and
(e) comparing the score to a cut-off value that is predictive of a disease or symptom in order to determine the presence of HCC in the subject.

13. The method of claim 12, wherein the amount of each of the Cas-L activity, Tr-L activity, and Ch-L activity are assayed in a sample from the subject.

14. The method of claim 13, wherein the amount of at least one of APP and DCP are assayed in a sample from the subject.

15. The method of claim 12, wherein the score is determined using the algorithm:
Score=y/(1−y)
wherein,
y=exp[−X+(C₁×Age)+(C₂×Gender)+(C₃×DCP)−(C₄×Ch-L)+(C₅×Cas-L/p)+(C₆×Tr-L/p)+(C₇×AFP)]
wherein X is from 16.7293 to 20.4471 inclusive;
C₁ is from 0.2027 to 0.2479 inclusive;
C₂ is from 3.9908 to 4.8778 inclusive;
C₃ is from 0.97557 to 1.1681 inclusive;
C₄ is from 23.5331 to 28.7627 inclusive;
C₅ is from 3.0299 to 3.7033 inclusive;
C₆ is from 0.0558 to 0.0682 inclusive;
C₇ is from 0.1534 to 0.1876 inclusive;
and wherein, age is provided in years; male gender=1, female gender=0; AFP is reported in ng/mL; DCP is reported in ng/mL; normalized Cas-L (Cas-L/p) is reported in pmol product/sec/pg proteasomal protein; normalized Tr-L (Tr-L/p) is reported in pmol product/sec/pg proteasomal protein; and Ch-L is reported in pmol product/sec/mL.

16. The method of claim 15, wherein
X is about 18.5882;
C₁ is about 0.2253;
C₂ is about 4.4343;
C₃ is about 1.0619;
C₄ is about 26.1479;
C₅ is about 3.3666;
C₆ is about 0.062;
C₇ is about 0.1705.

17. The method of claim 15 wherein the cut-off value is about 0.5 and a score less than about 0.5 is indicative of the absence of HCC in the subject.

18. The method of claim 15, wherein the cut-off value is about 0.5 and a score greater than or equal to about 0.5 is indicative of HCC in the subject.

19. The method of claim 12, wherein the sample is serum or plasma.

20. The method of claim 21, wherein the score is used for the choice of a suitable treatment for the subject.

21. A method for monitoring progression of hepatocellular carcinoma (HCC) in a subject, the method comprising:
(a) providing a first sample from the subject;
(b) assaying in the sample the amount of
(i) enzymatic activity from one or more of Ch-L, Tr-L, and Cas-L,
(ii) one or more of AFP, AFP-L3, DCP, and ubiquitin,
(iii) proteasomal protein and normalizing at least one enzymatic activity determined in step (b)(i);
(c) determining a score for the subject based on the assayed levels in (b);
(d) comparing the score to a cut-off score that is predictive of HCC in order to determine the extent of HCC in the subject;
(e) providing a second sample from the subject, wherein the second sample is obtained after the first sample;
(f) repeating steps (b) to (d) to determine the extent of HCC as indicated by the second sample; and
(g) comparing the extent of HCC indicated by the first sample to the extent of HCC indicated by the second sample, wherein a higher extent of HCC in the second sample in comparison to the first sample indicates progression of HCC or a lesser extent of HCC in the second sample in comparison to the first sample indicates regression of HCC.

22. The method of claim 21, the score is determined using the algorithm:
Score=y/(1+y)
wherein,
y=exp[−X(C₁×Age)+(C₂×Gender)+(C₃×DCP)−(C₄×Ch-L)+(C₅×Cas-L/p)+(C₆×Tr-L/p)+(C₇×AFP)]
wherein X is from 16.7293 to 20.4471 inclusive;
C₁ is from 0.2027 to 0.2479 inclusive;
C₂ is from 39908 to 4.8778 inclusive;
C₃ is from 0.97557 to 1.1681 inclusive;
C₄ is from 23.5331 to 28.7627 inclusive;
C₅ is from 3.0299 to 3.7033 inclusive;
C₆ is from 0.0558 to 0.0682 inclusive;
C₇ is from 0.1534 to 0.1876 inclusive;
and wherein, age is provided in years; male gender=1, female gender=0; AFP is reported in ng/mL; DCP is reported in ng/mL; normalized Cas-L (Cas-L/p) is reported in pmol product/sec/pg proteasome; normalized Tr-L (Tr-L/p) is reported in pmol product/sec/pg proteasome; and Ch-L is reported in pmol product/sec/mL.

23. The method of claim 22, wherein
X is about 18.5882;
C₁ is about 0.2253;
C₂ is about 4.4343;
C₃ is about 1.0619;
C₄ is about 26.1479;
C₅ is about 3.3666;
C₆ is about 0.062; and
C₇ is about 0.1705.
